(19) United States
(12) Patent Application Publication
(10) Pub. No.: US 2010/0129908 A1
(43) Pub. Date: May 27, 2010

(54) SPACED PROJECTION SUBSTRATES AND DEVICES FOR CELL CULTURE

(76) Inventors: Ye Fang, Painted Post, NY (US); Yi-Cheng Hsieh, Yi-Lan (TW); Ling Huang, Crescent (SG); Ying Wei, Painted Post, NY (US)

Correspondence Address: CORNING INCORPORATED, SP-TI-3-1, CORNING, NY, 14831, US

(21) Appl. No.: 12/622,079

(22) Filed: Nov. 19, 2009

Related U.S. Application Data
(60) Provisional application No. 61/116,787, filed on Nov. 21, 2008.

(51) Int. Cl. C12N 5/071

(52) U.S. Cl. 435/370; 435/395; 435/396

(57) ABSTRACT

An article for culturing cells includes a substrate on which cells can be cultured. The substrate has a base surface. An array of projections extends from the base surface. The projections have a height of about 1 micrometer to about 100 micrometers, and have a gap distance along the major surface from center to center between neighboring projections of about 10 micrometers to 80 micrometers. A plurality of arrays of projections may extend from the surface with gaps in the base surface between the arrays. Hepatocytes cultures on such microprojection array substrates maintained in vivo like morphology and membrane polarity. Hepatocytes co-cultured with helper cells on such substrates tended to grow in the area of the arrays, while the helper cells tended to grow in the areas between the arrays.

### CLAIMING BENEFIT OF PRIOR FILED U.S. APPLICATION

This application claims the benefit of U.S. Provisional Application Ser. No. 61/116,787, filed on Nov. 21, 2008. The content of this document and the entire disclosure of any publication, patent, or patent document mentioned herein is incorporated by reference.

### FIELD

The present disclosure relates to apparatus for culturing cells; more particularly to cell culture apparatuses having structured protrusions for facilitating desired characteristics of cells cultured on the apparatus.

### BACKGROUND

Cells cultured on flat cell culture ware often provide artificial two-dimensional sheets of cells that may have significantly different morphology and function from their in vivo counterparts. Cultured cells are crucial to modern drug discovery and development and are widely used for drug testing. However, if results from such testing are not indicative of responses from cells in vivo, the relevance of the results may be diminished. Cells in the human body experience three dimensional environments completely surrounded by other cells, membranes, fibrous layers, adhesion proteins, etc. Thus, substrates that prompt cultured cells to have in vivo-like morphology and function are desirable.

Advanced cell culture and tissue engineering generally utilizes three-dimensional polymeric scaffolds to reflect normal cell morphology and behavior for more realistic cell culture. There are wide ranges of scaffold substrates available and used to serve as synthetic extracellular matrices (ECMs). These synthetic ECM scaffolds are generally open, porous and exogenous and are typically fabricated from biocompatible, biodegradable polymers. However, such synthetic ECM substrates often lead to great variability in morphology and function of cultured cells from well to well and from culture to culture due to variability in the structure of the ECM scaffolds.

Tissue engineering employs exogenous three-dimensional extracellular matrices (ECMs) to engineer new natural tissues from isolated cells. The loss or failure of an organ or tissue is one of the most severe human health problems. Tissue or organ transplantation is a standard therapy to treat these patients, but this is severely limited by a donor shortage. Other available therapies to treat these patients include surgical reconstruction (e.g. heart), drug therapy (e.g. insulin for a malfunctioning pancreas), synthetic prostheses (e.g. polymeric vascular prostheses) and mechanical devices (e.g. kidney dialysis). Although these therapies are not limited by supply, they do not replace all functions of a lost organ or tissue and often fail in the long term. Tissue engineering has emerged as a promising approach to treat the loss or malfunction of a tissue or organ without the limitations of current therapies. This approach has a foundation in the biological observation that dissociated cells will reassemble in vitro to form structures that resemble the original tissue when provided with an appropriate environment. The exogenous ECMs employed in tissue engineering are designed to bring the desired cell types into contact in an appropriate three-dimensional environment, and also provide mechanical support until the newly formed tissues are structurally stabilized. However, the variable structure of such ECMs may result in too much variability in resulting engineered tissues for practical application.

### BRIEF SUMMARY

The present disclosure describes the use of structurally geometrically defined smart substrates employing spaced projections for cell culture. In one disclosed embodiment, structurally regulated adhesion and intercellular interaction results in cultured hepatocyte cells displaying in vivo-like morphology and functions. The well-defined geometries of the smart substrates can provide physical constraints of cell spreading, adhesion and growing, guide intercellular interaction and communications, and can lead to controlled size and dimensions of cultured cell clusters.

In various embodiments, an article for culturing cells includes a substrate having a base surface on which cells can be cultured. The base surface of the substrate is the top surface of a bottom of a well of the article. The article further includes an array of projections extending from the base surface. The projections have a height of between about 1 micrometer and about 100 micrometers. The projections preferably have a height of between about 1 micrometer and about 10 micrometers, A gap distance along the base surface from center to center between neighboring projections is between about 10 micrometers and about 80 micrometers. Such cell culture articles are shown herein to be effective for restoring membrane polarity and supporting the in vivo-like biological functions of cultured hepatocytes.

In various embodiments, an article has a plurality of arrays of such projections extending from the base surface. A gap distance along the base surface may exist between the arrays. Such cell culture articles are shown herein to support cell co-culture of hepatocytes and helper cells, wherein the hepatocyte growth primarily occurs in areas occupied by the arrays and helper cells mainly grow on the base surface in the areas between the arrays of projections.

A variety of methods for culturing hepatocytes and co-culturing hepatocytes with helper cells are also described herein. The methods include culturing hepatocytes on structured surfaces, such as those described above, that provide for restoring of hepatocyte membrane polarity or for gaining of hepatocyte metabolic function. The methods include co-culturing hepatocytes with helper cells on structured surfaces, such as those described above, that provide for segregation of hepatocytes and helper cells on the structured surfaces and for guiding the interactions between the hepatocytes and the helper cells. Such segregation may be beneficial for maintaining in vivo-like characteristics of the cultured hepatocytes.

### BRIEF DESCRIPTION OF THE DRAWINGS

FIG. 1 is perspective view of a schematic cell culture article having an array of structured projections extending from a base surface of the article.

FIG. 2 is a perspective view of a sectioned schematic cell culture article having an array of structured projections extending from a base surface of the article.

FIG. 3A is a top down view of a schematic illustration of cells cultured between projections of an array.

FIG. 3B is a side view of a schematic illustration of cells cultured on a base surface of a substrate of a cell culture article between projections extending from the surface.

FIG. 4 is a top-down view of a schematic cell culture article having an array of structured projections extending from a base surface of the article.

FIGS. 5A-B are perspective views of schematic diagrams of projections.

FIGS. 6 and 7A are schematic top-down views of cell culture articles having arrays of structured projections extending from a base surface of the article.

FIG. 7B is a close-up of a schematic top-down view of a cell culture well of FIG. 7A.

FIGS. 8 and 9A-C are flow diagrams of methods for culturing cells on cell culture articles having an array of structured projections extending from a base surface of the article.

FIG. 10 is a schematic illustration of hepatocytes in vivo, showing polarity of the hepatocytes and their relationship with sinusoidal cells.

FIGS. 11A-D are microscopic images of hepatocytes cultured on articles having arrays of projections extending from a base surface of the article.

FIGS. 12A-D are microscopic images of hepatocytes cultured on articles having arrays of projections extending from a base surface of the article.

FIGS. 13A-D are microscopic images of hepatocytes cultured on articles having arrays of projections extending from a base surface of the article.

FIGS. 14A-D are microscopic images of hepatocytes cultured on articles having arrays of projections extending from a base surface of the article.

FIGS. 15A-F are microscopic images of hepatocytes cultured on articles having arrays of projections extending from a base surface of the article.

FIGS. 16A-B are microscopic images of hepatocytes cultured on articles having arrays of projections extending from a base surface of the article.

FIGS. 17A-D are microscopic images of hepatoctes cultured on articles having arrays of projections extending from a base surface of the article.

FIGS. 18A-B are graphs showing cell growth and viability testing of hepatocyte hepG2C3A cells on uncoated and collagen I coated articles having arrays of projections extending from a base surface of the article, in comparison with those on collagen I coated tissue culture microplates.

FIG. 19 is a light microscopic image of NIH3T3 fibroblast cells on oxidized PDMS microprojection array substrate.

FIG. 20 is a light microscopic image of co-culture of NIH3T3 fibroblast cells and hepG2C3A cells on oxidized PDMS microprojection array substrate.

FIG. 21 is a light microscopic image of co-culture of NIH3T3 fibroblast cells and hepG2C3A cells on oxidized PDMS microprojection array substrate.

FIG. 22 is a graph of rifampin-induced CYP3A4 enzyme activity of HepG2C3A cells co-cultured with NIH3T3 cells on the oxidized PDMS microprojection substrates or a collagen-coated control substrate.

FIG. 23A-B are light microscopic images of hepG2C3A cells on an oxidized PDMS microprojection array substrate after 28 days of culture.

FIG. 24A-B are light microscopic images of hepG2C3A cells on a collagen I coated oxidized PDMS microprojection array substrate after 28 days of culture.

FIG. 25 is a graph of rifampin-induced CYP3A4 enzyme activity of HepG2C3A cells cultured on different oxidized PDMS microprojection substrates, in comparison with the cells on collagen I coated TCT (tissue culture treated) surfaces.

FIG. 26 is a graph of expression of 10 CYP genes in cryopreserved primary hepatocytes without any further culture.

FIG. 27 is a graph of expression of 10 CYP genes in primary hepatocytes cultured on different PDMS projection array substrates, according to the present invention.

FIG. 28 is a graph of expression of 10 transporter genes in cryopreserved primary hepatocytes without any further culture.

FIG. 29 is a graph of expression of 10 transporter genes in primary liver cells cultured on different PDMS projection array substrates, according to the present invention.

The drawings are not necessarily to scale. Like numbers used in the figures refer to like components, steps and the like. However, it will be understood that the use of a number to refer to a component in a given figure is not intended to limit the component in another figure labeled with the same number. In addition, the use of different numbers to refer to components is not intended to indicate that the different numbered components cannot be the same or similar.

### DETAILED DESCRIPTION

In the following detailed description, reference is made to the accompanying drawings that form a part hereof, and in which are shown by way of illustration several specific embodiments of devices, systems and methods. It is to be understood that other embodiments are contemplated and may be made without departing from the scope or spirit of the present disclosure. The following detailed description, therefore, is not to be taken in a limiting sense.

All scientific and technical terms used herein have meanings commonly used in the art unless otherwise specified. The definitions provided herein are to facilitate understanding of certain terms used frequently herein and are not meant to limit the scope of the present disclosure.

As used in this specification and the appended claims, the singular forms “a”, “an”, and “the” encompass embodiments having plural referents, unless the content clearly dictates otherwise. As used in this specification and the appended claims, the term “or” is generally employed in its sense including “and/or” unless the content clearly dictates otherwise.

As used herein, “have”, “having”, “include”, “including”, “comprise”, “comprising” or the like are used in their open ended sense, and generally mean “including, but not limited to.”

Any direction referred to herein, such as “top,” “bottom,” “left,” “right,” “upper,” “lower,” and other directions and orientations are described herein for clarity in reference to the figures and are not to be limiting of an actual device or system or use of the device or system. Devices or systems as described herein may be used in a number of directions and orientations.

The present disclosure describes, inter alia, cell culture apparatus geometrically defined substrates employing spaced projections for cell culture. The well-defined geometries of the substrates and projections can provide physical constraints of cell spreading, adhesion and growing, guide intercellular interactions and communications, and can lead to controlled size and dimensions of cultured cell clusters. The defined geometries can result in more realistic cellular interaction, biology and function.

Any suitable cell culture article may be modified to employ structured surfaces as described herein. For example, single and multi-well plates, such as 6, 12, 96, 384, and 1536 well plates, jars, petri dishes, flasks, beakers, plates, roller bottles, slides, chambered and multichambered culture slides, channeled or microchanneled (i.e., an enclosed channeled or microchanneled device having the microstructures on at least one surface) culture devices, tubes, cover slips, cups, spinner bottles, perfusion chambers, bioreactors, and fermenters may include a structure surface in accordance with the teachings provided herein. Such articles may be fabricated from any suitable base material, such as glass materials including soda-lime glass, pyrex glass, vycor glass, quartz glass; silicon; plastics or polymers, including dendritic polymers, such as poly(vinyl chloride), poly(vinyl alcohol), poly(methyl methacrylate), poly(vinyl acetate-maleic anhydride), poly(dimethylsiloxane) monomethacrylate, cyclic olefin polymers and copolymers including copolymers of norbornene and ethylene, fluorocarbon polymers, polystyrenes, polypropylene, polyethyleneimine; copolymers such as poly(vinyl acetate-co-maleic anhydride), poly(styrene-co-maleic anhydride), polysaccharide, polysaccharide peptide, poly(ethylene-co-acrylic acid) or derivatives of these or the like.

In alternative embodiments, a polymeric substrate having spaced projections can be used as a carrier for cell culture, wherein the substrate is suspended in cell culture medium, and the cells become adherent onto and grow on the substrate. The polymeric substrate having spaced projects can be deformed (e.g., folded), or planar. The polymeric substrate having spaced projections or a plurality of arrays of projections is preferably a thin sheet with a thickness less than 100 microns. The thin polymeric sheet having spaced projections or arrays or a plurality of arrays of projections can be in any shape, such as regular, or irregular.

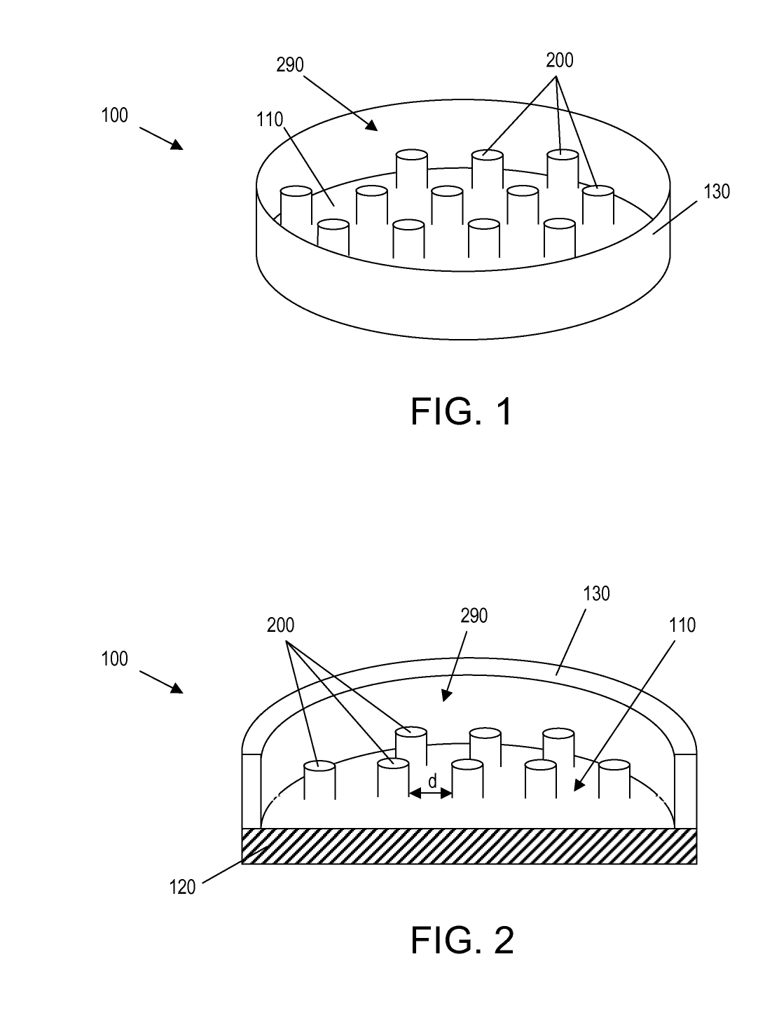

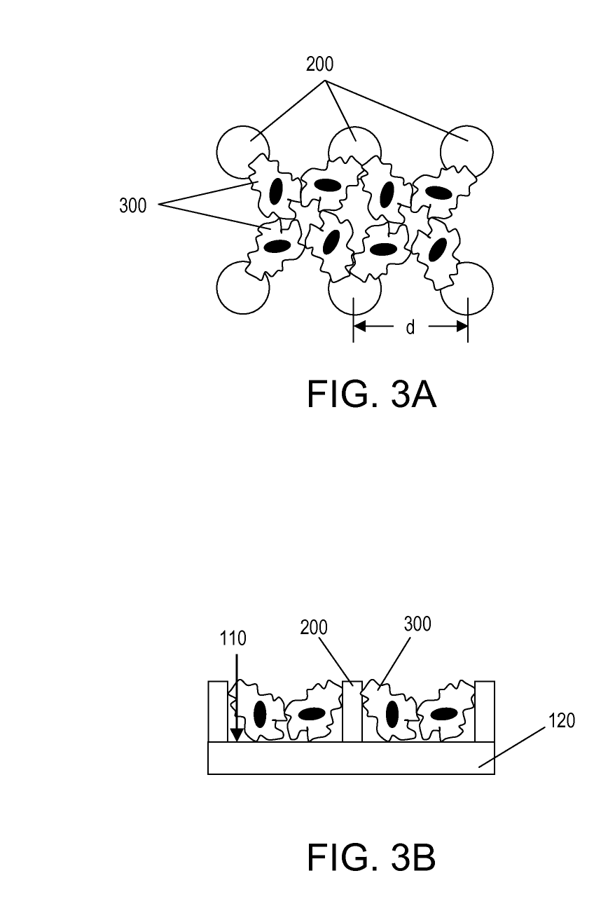

With reference to FIGS. 1-2, representative cell culture articles 100 are shown. The cell culture articles 100 each have an array 290 of projections 200 extending from a base surface 110 of a substrate 120 of the cell culture articles 100. The cell culture articles have a side wall 130. The base surface 110 and array 290 of projections 200 define a structured and highly reproducible three-dimensional geometry for culturing cells. Each projection 200 of an array 290 has defined geometric dimensions that are reproducible to a degree commensurate with the reproducibility of the processes employed to form the projections 200. The projections 200 are spaced apart by distance (d) to allow sufficient room for cells to be cultured on the base surface 110 of the cell culture article 100 with at least a portion of the cells contacting a projection 200. For example and with reference to FIGS. 3A-B, in which schematics of top-down (3A) and side (3B) views of cells 300 cultured on articles having spaced projections 200 are shown, projections 200 are placed apart such that at least some cells 300 may contact the base surface 110 of the substrate 120 and contact a projection 200. Clusters of cells 300 may be formed within the gaps between projections 200. The number of projections 200 in an array and the gap distance (d) between neighboring projections 200 can be controlled to control the number of cell clusters and the number of cells in a cluster. Such control may provide advantages relative to culturing on more traditional substrates. For example, by controlling the number of cells in a cluster by controlling the spacing between projections 200, more ready reliable normalization of results from studies performed on the cultured cells relative to cells cultured on more traditional articles is provided, where the number of cells in a give area or throughout the culture surface can be quite variable. In addition, the gap distance between neighboring projections may be varied to maximize the cell culture results. For example, one cell type may culture more favorably in a cell culture apparatus having a gap distance between pillars that is different from the gap distance that provides the most favorable cell culture environment for a different cell type. Additional benefits of using such a device for cell culture include the free access of cells to nutrition and/or the ability to expel waste generated by the cells, due to the guided contacts of the cells with both the base surface and the side of the projections.

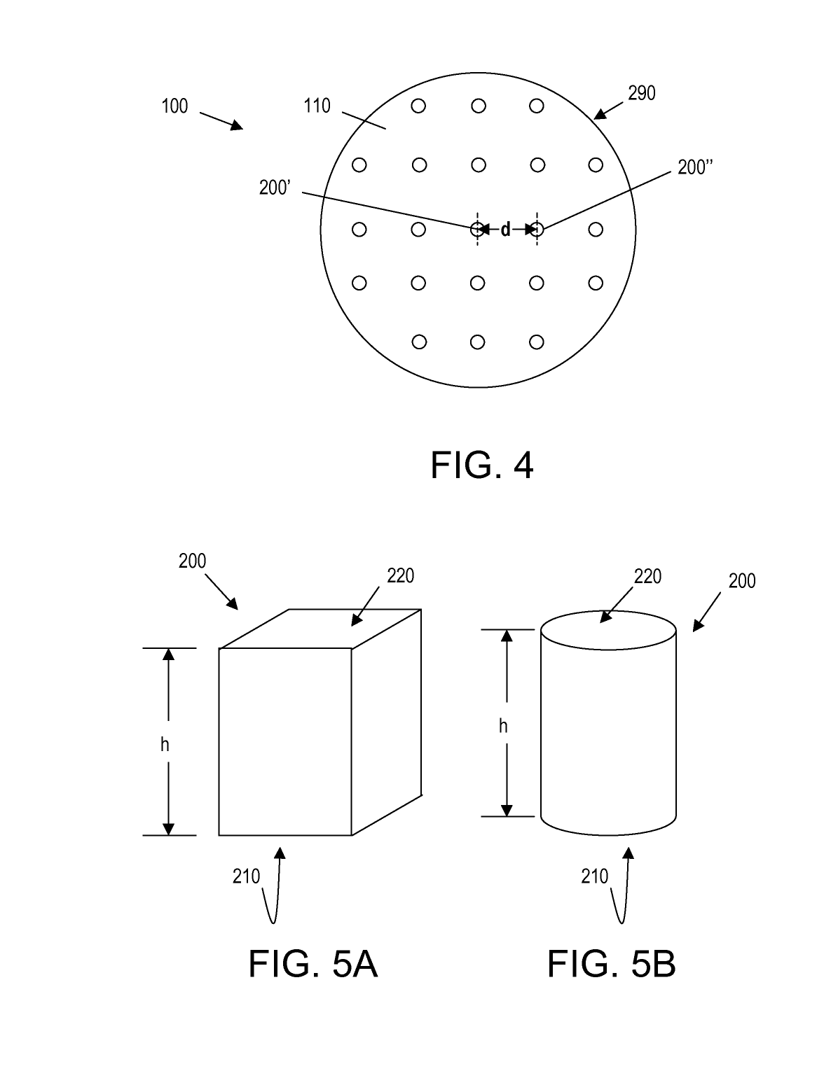

Referring now to FIG. 4, which shows a top-down view of a schematic of a representative cell culture article 100, the space from the center of a projection 200′ of an array 290 to the center of its nearest neighboring projection 200″ is shown as distance (d) (or gap distance). In embodiments, the distance d is greater than about 10 micrometers. In various embodiments, the distance along the major surface 110 from the geometric center of a projection 200′ (on the base surface 110) to the geometric center (on the base surface 110) of the nearest projection 200″ is between about 10 micrometers and about 80 micrometers, between about 10 micrometers and about 50 micrometers, between about 10 micrometers and about 30 micrometers, or about 20 micrometers, or between about 30 micrometers and about 70 micrometers. The distance d between each projection 200 and its nearest neighboring projection 200 need not be same for all projections, provided that a sufficient number of projections are spaced at least 10 micrometers from their nearest neighbor (from geometric center to geometric center). In some embodiments, all of the projections 200 in an array 290 are spaced at least 10 micrometers from their nearest neighbor (from center to center).

While the projections depicted in FIGS. 1-4 and other figures presented herein are in the form of cylinders, it will be understood that the projections may be of any suitable shape, such as a cubiod, a pyramid, a cone, or the like. The projections can be solid, or porous with nanometer scale porosity or microscale porosity. Depending on the materials used, the mechanical properties of the projections can vary significantly, and thus can be fine tuned for culturing specific types of cells.

Referring now to FIGS. 5A-B, each projection 200 in an array has a height h. The height h can be measured as the orthogonal distance from the base surface of the cell culture article from which projection 200 extends to the point furthest from the base surface. The height h of each projection 200 in an array may be the same or different. The height h of a projection 200 in an array may be greater than about 1 micrometer. In various embodiments, the height h of the projection 200 is between about 1 micrometer to about 100 micrometers, between about 1 micrometer and about 50 micrometers, between about 5 micrometer and about 25 micrometers, between about 2 micrometer and about 10 micrometers, or about 5 micrometers.

Still with reference to FIGS. 5A-B, each projection 200 of an array has surface 210 in contact with, or extending from, the base surface of the article, and an opposing surface 220 of the projection 200, which surfaces 210, 220 may have the same or different surface areas depending on the overall geometry of the projection 200. Surfaces 210, 220 may have any suitable surface area. In many embodiments, the surface area of both surface 220 and surface 210 are greater than about 1 square micrometer. In various embodiments, the surface area of surface 220 is between about 1 square micrometer and about 500 square micrometers, between about 5 square micrometers and about 250 square micrometers, between about 5 square micrometers and about 100 square micrometers, and between about 25 square micrometers and about 100 square micrometers. In some embodiments, projection 200 is cylindrically shaped, e.g. as depicted in FIG. 5B, and surface 220 has a diameter of between about 1 micrometer and 25 micrometers, or between about 5 micrometers and about 15 micrometers. In additional embodiments, the projection 200 is rectangular (as depicted in FIG. 5A), cuboidal, conical, rhomboid, or any other geometrical shape.

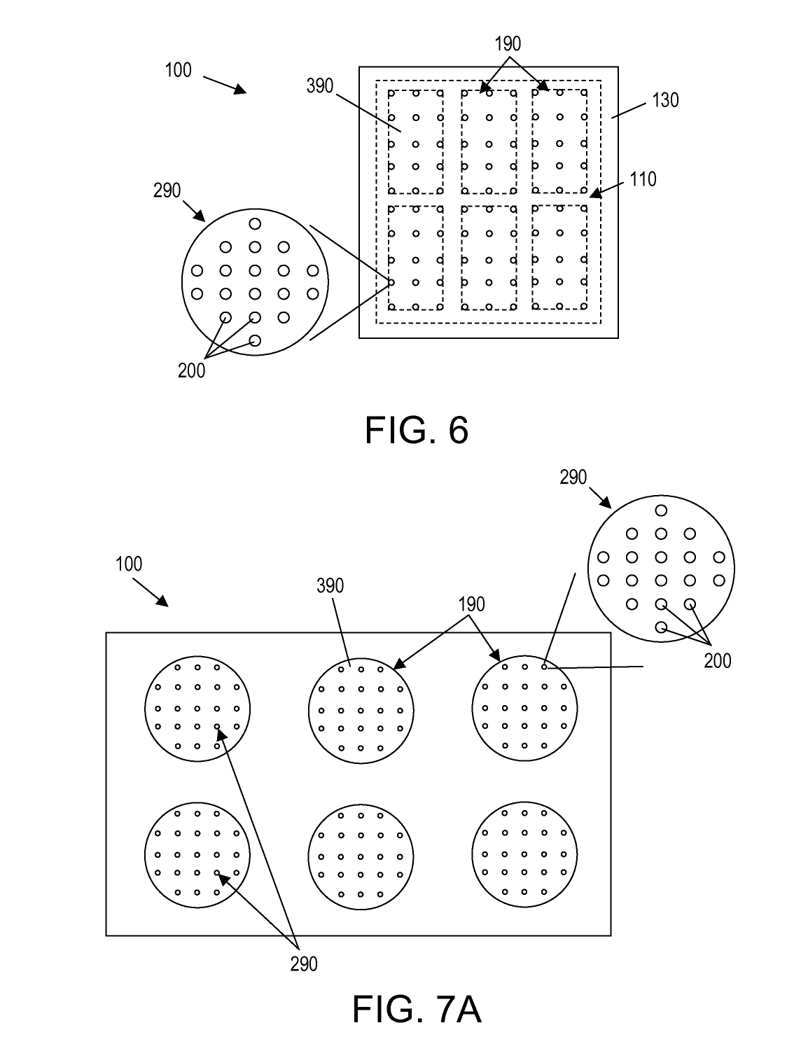

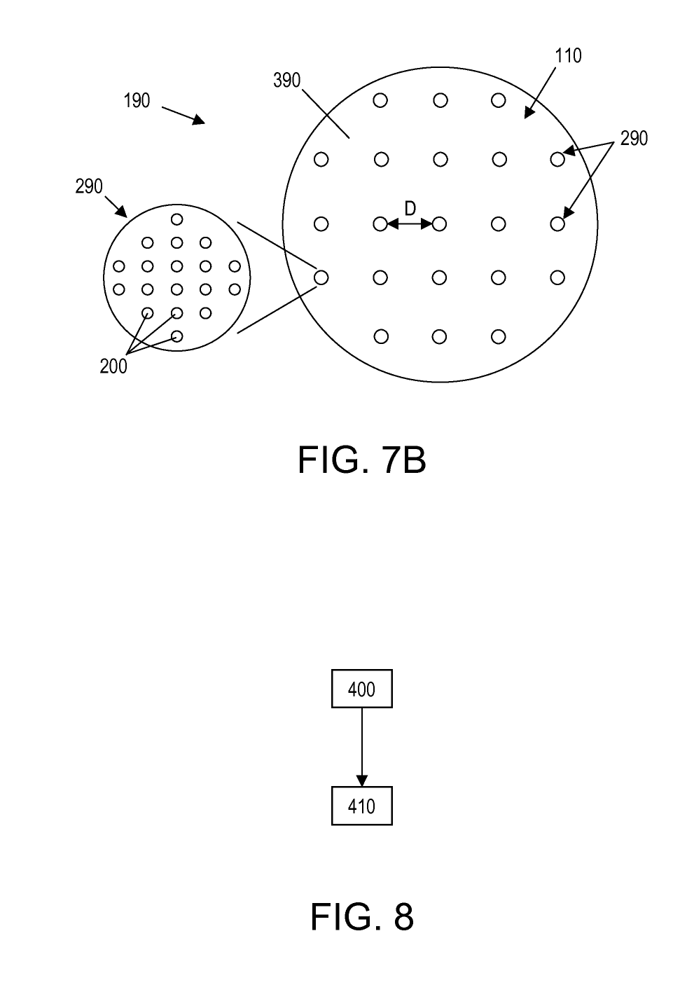

Referring now to FIGS. 6-7, top-down views of schematic cell culture articles 100 are shown. The article 100 may include one or more macroarrays 190, each having a plurality of microarrays 290. The projections 200 of the microarrays 290 (and the gap distance between projections) may be as described above with regard to FIGS. 1-5. The macroarrays 190 may include any suitable pattern of microarrays 290. In the depicted embodiments, the macroarrays 190 are identical to the extent that a process for producing the macoarrays 190 is reproducible.

In the embodiment depicted in FIG. 6, the article 100 has a side wall 130 defining a well having a base surface 110 on which cells may be cultured, The projections 200 of the microarrays 290 extend from the base surface 110 (e.g., as described with regard to FIGS. 1-5). In the embodiment depicted in FIG. 7A, the article 100 has six macroarrays 190, each within a well of the culture article 100, with twenty one microarrays 290 per macroarray 190. Each well has a base surface 110 on which cells may be cultured (see FIG. 7B, which is a close-up view of a single well of the article 100 shown in FIG. 7A).

Still referring to FIGS. 6-7, the arrays 190 may occupy any suitable surface area of the culture base surface 110 defined by a well or other suitable culture surface of the article 100. In various embodiments, each array 190 occupies a surface area of between about 10,000 square micrometers and about 25,000,000 square micrometers, between about 10,000 square micrometers and about 300,000 square micrometers, or between about 100,000 square micrometers and about 300,000 square micrometers. In some embodiments, e.g. as depicted in FIGS. 6-7, the microarrays 290 of projections occupy a generally circular surface area of a well or other suitable culture surface of the article 100. Such circular arrays may have any suitable surface area. For example, the diameter of a circular microarray 290 may be between about 100 micrometers and about 500 micrometers. The reference numeral 390 depicted in FIGS. 6, 7A, and 7B denotes space on the cell culture surface 110 between microarrays 290.

As shown in FIG. 7B, neighboring arrays 290 may be separated by an array distance D along the base surface 110. Like the gap distance and number of projections in an array 290, the number of arrays 290 and the array distance D between neighboring arrays 290 on a given culture surface 110 can be varied to control culture conditions. For example and as described in the Examples below, helper cells co-cultured with hepatocytes tend to segregate towards the spaces 390 between microarrays 290, while the hepatocytes tend to segregate towards the spaces occupied by the microarrays 290 of projections 200. Thus, by controlling the array distance D between microarrays 290 on a culture surface 110 or by controlling the relative area of the base surface 110 occupied by the microarrays 290 relative to the total surface area of the base surface 110, the relative surface area for culturing hepatocytes to surface area for culturing helper cells may be controlled. With different culture systems and different cell types, spacing and numbers of microarrays 290 may be varied advantageously.

The array distance D between nearest neighboring arrays 290 may be any suitable distance. For example, the array distance D may be between about 10 micrometers and about 1000 micrometers, between about 25 micrometers and about 500 micrometers, or between about 50 micrometers and about 250 micrometers. Similarly, arrays 290 may occupy any suitable percentage of the surface area of a base surface 110 for culturing cells. For example, arrays 290 may occupy between about 10% and about 100% of the surface area of a base surface 110, between about 25% and about 75% of the surface area of a base surface 110, or between about 40% and about 60% of the surface area of a base surface 110.

A close-up view of a selected array from each of FIGS. 6, 7A, and 7B is shown in the respective figure to more clearly show that in embodiments each array 290 may include a plurality of structurally defined projections 200.

An array may be formed via any suitable technique. For example, an array may be formed via a master, such as a silicon master. The master may be fabricated from silicon by proximity U.V. photolithography. By way of example, a thin layer of photoresist, an organic polymer sensitive to ultraviolet light, may be spun onto a silicon wafer using a spin coater. The photoresist thickness is determined by the speed and duration of the spin coating. After soft baking the wafer to remove some solvent, the photoresist may be exposed to ultraviolet light through a photomask. The mask's function is to allow light to pass in certain areas and to impede it in others, thereby transferring the pattern of the photomask onto the underlying resist. The soluble photoresist is then washed off using a developer, leaving behind a protective pattern of cross-linked resist on the silicon. At this point, the resist is typically kept on the wafer to be used as the topographic template for molding the stamp. Alternatively, the unprotected silicon regions can be etched, and the photoresist stripped, leaving behind a wafer with patterned silicon making for a more stable template. The lower limit of the features on the structured substrates is dictated by the resolution of the fabrication process used to create the template. This resolution is determined by the diffraction of light at the edge of the opaque areas of the mask and the thickness of the photoresist. Smaller features can be achieved with extremely short wavelength UV light (˜200 nm). For submicronic patterns (e.g. etch depths of about 100 nanometers), electron beam lithography on PMMA (polymethylmetacrylate) may be used. Templates can also be produced by micromachining, or they can be prefabricated by, e.g., diffraction gratings.

To enable simple demoulding of the master, an anti-adhesive treatment may be carried out using silanization in liquid phase with OTS (octadecyltrichlorosilane) or fluorinated silane, for example. After developing, the wafers may be vapor primed with fluorinated silane to assist in the subsequent removal of the array of projections. Examples of fluorinated silane that may be used include, but are not limited to, (tridecafluoro-1,1,2,2-tetrahydroctyl) trimethoxysilane, and tridecafluoro-1,1,2,2-tetrahydrooctyl) triethoxysilane.

Projections may be made of any suitable polymeric material or inorganic material. Suitable inorganic materials include glass, silica, silicon, metal, or the like. Suitable polymeric materials include poly(dimethylsiloxane) (PDMS), a sol-gel, or other cell culture compatible polymer. Examples of suitable sol gels include sol gels formed through the hydrolysis of tetraethyl orthosilicate (TEOS) under acidic conditions. Other cell culture compatible polymers include polyesters of naturally occurring α-hydroxy acids, polyglycolic acid (PGA), poly(-lactic acid) (PLLA) and copolymers of poly(lactic-co-glycolic acid) (PLGA), amino-acid-based polymers, a polysaccharide, or polystryrene. The materials for forming projections may be chosen based on desired mechanical, cell-interacting, or other properties for optimizing cell culture for distinct types of cells.

Projections may be made of the same material as the substrate from which they extend or may be made of different material from the substrate. The projections or substrate can be porous, nano-porous, microporous, or macroporous. Projections or substrates may be treated or coated to impart a desirable property or characteristic to the treated or coated surfaces. Examples of surface treatments often employed for purposes of cell culture include corona or plasma treatment. In various embodiments, projections or substrate surfaces are coated with extracellular matrix (ECM) materials, such as naturally occurring ECM proteins or synthetic ECM materials. The type of EMC selected may vary depending on the desired result and the type of cell being cultures, such as a desired phenotype of the culture cells. Examples of naturally occurring ECM proteins include fibronectins, collagens, proteoglycans, and glycosaminoglycans. Examples of synthetic materials for fabricating synthetic ECMS include polyesters of naturally occurring α-hydroxy acids, poly(DL-lactic acid), polyglycolic acid (PGA), poly(-lactic acid) (PLLA) and copolymers of poly(lactic-co-glycolic acid) (PLGA). Such thermoplastic polymers can be readily formed into desired shapes by various techniques including molding such as injection molding, extrusion and solvent casting. Amino-acid-based polymers may also be employed in the fabrication of an ECM for coating a projection or substrate. For example, collagen-like, silk-like and elastin-like proteins may be included in an ECM. In various embodiments, an ECM includes alginate, which is a family of copolymers of mannuronate and guluronate that form gels in the presence of divalent ions such as Ca²⁺. Any suitable processing technique may be employed to fabricate ECMs from synthetic polymers. By way of example, a biodegradable polymer may be processed into a fiber, a porous sponge or a tubular structure.

One or more ECM material may be used to coat the projections or substrates. For example, in embodiments, cell adhesion factors, such as polypeptides capable of binding integrin receptors including RGD-containing polypeptides, or growth factors can be incorporated into ECM materials to stimulate adhesion or specific functions of cells using approaches including adsorption or covalent bonding at the surface or covalent bonding throughout the bulk of the materials.

Cell culture articles having projection arrays as described above may be used to culture a variety of cells and may provide important three dimensional structures to impart desirable characteristics to the cultured cells. While cells of any type or combination of types (e.g., stem cells) may be cultured on such projection array substrates, additional detail will now be presented with regard to culturing hepatocytes on such substrates. As described in the Examples below, cell culture articles having structured projection arrays have been shown, for the first time, to result in cultured hepatocytes restoring their polarity and metabolic functions.

In vitro cultured hepatocytes are popular for drug metabolism and toxicity studies. However, hepatocytes cultured on conventional two-dimensional cell culture substrates rapidly loose their polarity and their ability to carry out drug metabolism and transporter functions. To improve the ability to maintain drug metabolism and transporter functions, hepatocytes have been cultured in well established in vitro models including (i) culturing on MATRIGELT™ (BD Biosciences), an animal derived proteineous matrix, and (ii) culturing in a sandwich culture system between two layers of ECM such as collagen. However, such systems suffer from significant drawbacks including the potential for contamination of the human hepatocytes due to animal origin of the MATRIGEL™ or ECM materials, complex molecular compositions, batch-to-batch variations and uncontrollable coating. Culturing hepatocytes on structured projection arrays as described herein may overcome one or more of the drawbacks of prior systems.

In various embodiments, functional hepatocytes may be cultured on a cell culture surface having an array of projections extending from a base surface, e.g. on a surface as described above with regard to FIGS. 1-7. With reference to FIG. 8, an embodiment of a method for culturing hepatocytes for maintaining polarity is depicted. As shown in the depicted flow diagram, hepatocytes are placed on a cell culture surface having a structured projection array extending from the surface (400) and the cells are cultured on the surface (410). In embodiments, culturing hepatocytes according to the methods shown in FIG. 8 restores the polarity of the hepatocytes, or maintains one or more functions of the hepatocyte. Any hepatocyte cell may be cultured in accordance with the method depicted in FIG. 8. For example, the hepatocytes to be cultured may be human HepG2 cells, human HepG2C3A cells, immortalized FaN4 cells, human primary liver cells, stem cell-derived hepatocytes, or the like, or combinations thereof.

In embodiments, the hepatocytes are cultured on an article having a substrate having a base surface and an array of projections extending from the base surface. In various embodiments, the projections have a height from about 1 micrometer to about 20 micrometer, and the gap distance (d; see, e.g. FIGS. 2, 3A, and 4) along the base surface from center to center between neighboring projections of the array is between about 10 micrometers and about 80 micrometers. In some embodiments, the hepatocytes are HepG2C3A cells and the gap distance along the major surface from center to center between neighboring projections is between about 15 micrometers and about 30 micrometers. In numerous embodiments, the cells are immortalized FaN4 cells and the gap distance along the major surface from center to center between neighboring projections is between about 15 micrometers and about 40 micrometers. In various embodiments, the hepatocytes are human primary liver cells and the gap distance along the major surface from center to center between neighboring projections is between about 30 micrometers and about 60 micrometers.

The hepatocytes may be seeded on the surface at any suitable density. Typically, hepatocytes are seeded at a density of between about 100 cells per square millimeter of surface area and about 5000 cells per square millimeter of surface area of the article or well. The seeding density can be optimized, based on culture conditions and duration. For example, for long term culture, the seeding density can be lower (e.g., 100 cells to 2000 cells per square millimeter of surface area of the article or well.

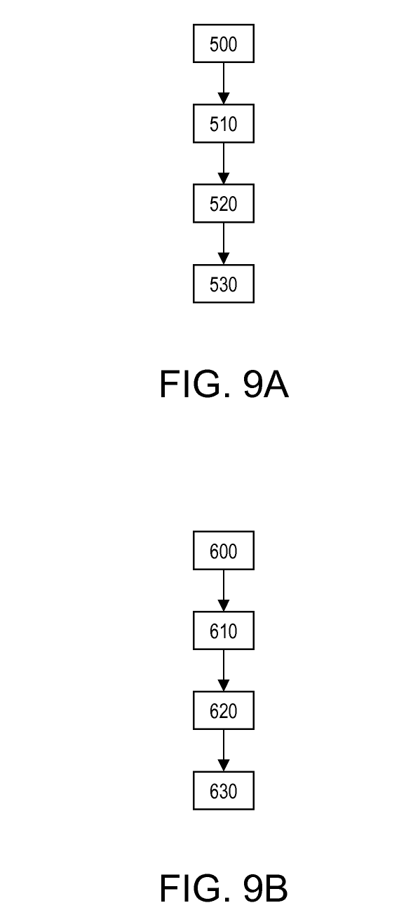

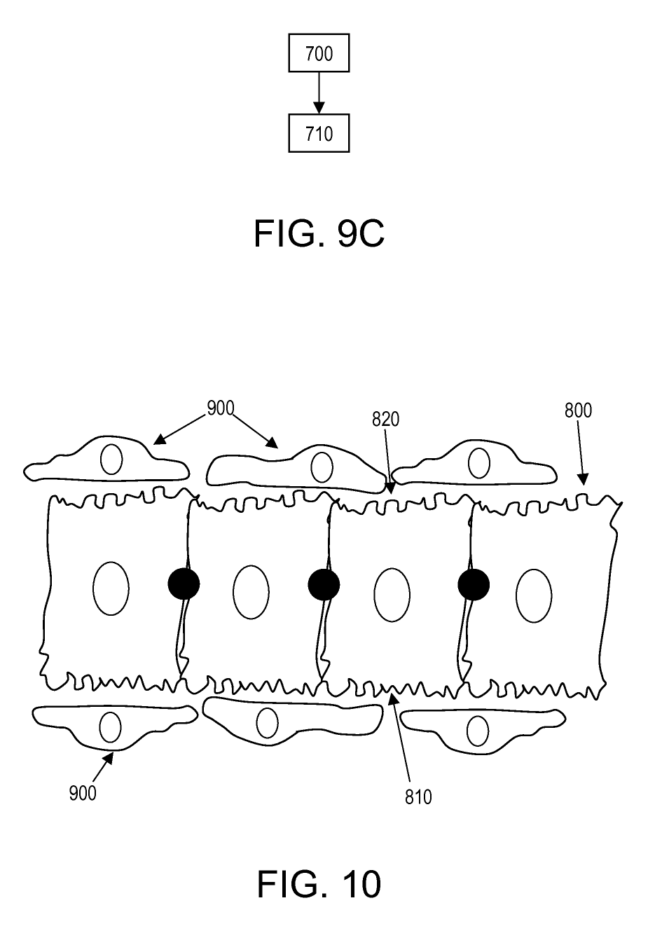

In various embodiments, hepatocytes are co-cultured with helper cells. Any suitable helper cell may be co-cultured with hepatocytes. Examples of suitable helper cells include fibroblasts such as NIH 3T3 fibroblasts, murine 3T3-J2 fibroblasts or human fibroblast cells; human or rat hepatic stellate cells; and Kupffer cells. With reference to FIGS. 9A-C, the helper cells may be added to the culture after (9A), before (9B), or at the same time (9C) as the hepatocytes. As indicated in FIG. 9A, hepatocytes may be placed on a cell culture surface having a structure array of projections extending from the surface (500) and cultured on the surface (510). Helper cells may then be placed on the cultured hepatocytes (520) and may be co-cultured with the hepatocytes (530). In embodiments, culturing hepatocytes with helper cells in the method depicted in FIG. 9A (9B or 9C) may restore the polarity of the hepatocytes or maintain one or more functions of the hepatocytes in culture. As indicated in FIG. 9B, helper cells may be placed on a cell culture surface having a structure array of projections extending from the surface (600) and cultured on the surface (610). Hepatocytes may then be placed on the cultured helper cells (620) and may be co-cultured with the helper cells (630). In embodiments, culturing hepatocytes with helper cells in the method depicted in FIG. 9A (9B or 9C) may restore the polarity of the hepatocytes or maintain one or more functions of the hepatocytes in culture. As indicated in FIG. 9C, hepatocytes and helper cells may be placed on a cell culture surface having a structure array of projections extending from the surface (700) and may be co-cultured together (710). In embodiments, culturing hepatocytes with helper cells in the method depicted in FIG. 9A (9B or 9C) may restore the polarity of the hepatocytes or maintain one or more functions of the hepatocytes in culture. While FIGS. 9A-B and the discussion above refer to placing helper cells on cultured hepatocytes (520) or placing hepatocytes on cultured helper cells (620), it will be understood that the helper cells or hepatocytes may be added to the culture before the hepatocytes or helper cells have covered the surfaces of the article, and thus at least some of the subsequently added helper cells or hepatocytes may be placed on the surface of the cell culture article rather than on the hepatocytes or helper cells. As described in more detail below in the Examples, helper cells co-cultured with hepatocytes tend to segregate towards areas between the arrays of projections, while the hepatocytes tend to segregate within the areas occupies by the arrays of projections. Such segregation provides for an arrangement of cells similar to in vivo cellular arrangements, where hepatocytes are generally grouped together.

The timing between seeding helper cells and hepatocyte cells can be fine tuned, and optimized. When the helper cells are seeded first in embodiments, may be the hepatocyte cells seeded one day afterwards. Conversely, when the hepatocytes are seeded first, the helper cells may be seeded after the hepatocytes restored their membrane polarity and/or metabolic functions (generally, 2-7 days). The seeding ratio between the helper cells and hepatocytes can be varied, depending on the substrate, culture conditions, and culture duration. For longer term culture (˜weeks), the helper cells seeded can be less than these short term culture (days).

Any suitable incubation time and conditions may be employed in accordance with the methods described herein. It will be understood that temperature, CO₂ and O₂ levels, culture medium content, and the like, will depend on the nature of the cells being cultured and can be readily modified. The amount of time that the cells are incubated on the surface may vary depending on the cell response being studied or the cell response desired. Prior to seeding cells, the cells may be harvested and suspended in a suitable medium, such as a growth medium in which the cells are to be cultured once seeded onto the surface. For example, the cells may be suspended in and cultured in serum-containing medium, a conditioned medium, or a chemically-defined medium. The optimal culture medium for each type of cells, such as recommended by American Tissue Cell Culture or other suppliers, can be used with or without modifications.

While much of the description provided herein relates to culturing hepatocytes on substrates having arrays of projections extending from the surface of the substrate, it will be understood that other cell types may be advantageously cultured on such substrates. Any cell type for which it may be beneficial to provide a structured and reproducible three dimensional environment may be advantageously cultured on substrates and articles as described herein. By way of example, the spacing and dimensions of projections and arrays may be controlled to affect the manner in which stem cells may differentiate.

In the following, non-limiting examples are presented, which describe various embodiments of the articles and methods discussed above.

### EXAMPLES

### I. Experimental Procedures

### A. Materials

Collagen I and MATRIGEL™ were purchased from BD Biosciences (Spears, Md.). Tissue culture treated polystyrene (TCT) 24 well microplates were purchased from Corning Inc. (Corning, N.Y.). Texas red labeled phalloidin (TR-Phalloidin) and all other chemicals were purchased from Sigma Chemical Co., St. Louis, Mo. Collagen I coated 24 well microplates were obtained from BD Biosciences.

### B. Fabrication of Silicon Master

A master for forming arrays was fabricated from silicon by proximity U.V. photolithography on a Si [100] wafer coated with positive resist (AZ 1529), and pattern transfer by deep reactive ion etching (1.4 μm deep). For submicronic patterns, electron beam lithography on PMMA (polymethylmethacrylate) was used instead of UV photolithography and the etch depth was limited to 100 nm. To enable simple demoulding of this master, an anti-adhesive treatment may be carried out using silanisation in liquid phase with OTS (octadecyltrichlorosilane) or fluorinated silane. After developing, the wafers were vapor primed with fluorinated silane to assist in the subsequent removal of the PDMS (polydimethylsiloxane). Examples of fluorinated silane that may be used include, but are not limited to, (tridecafluoro-1,1,2,2-tetrahydroctyl) trimethoxysilane, and tridecafluoro-1,1,2,2-tetrahydrooctyl) triethoxysilane.

### C. Fabrication of PDMS Projection Array Substrates

PDMS projection array substrates were formed by curing a PDMS pre-polymer solution containing a mixture (10:1 mass ratio) of PDMS oligomers and a reticular agent from Sylgard 184 Kit (Dow Corning) on the silicon master. The PDMS was thermally cured at 70° C. for 80 minutes. Flat PDMS substrates having projection arrays were formed by curing on silicon wafers that were vapor primed with fluorinated silane, and substrates of diameter of approximately 4 millimeters×4 millimeters were cut at each end of the cured PDMS projection array substrates with a scalpel.

PDMS is a silicone elastomer, (Sylgard 184, Dow Corning), that molds with very high fidelity to a patterned template. PDMS is a liquid prepolymer at room temperature due to its low melting point (about −50° C.) and glass transition temperature (about −120° C.). To fabricate PDMS structured substrates, the prepolymer is mixed with a curing agent, poured onto a template, and cured to crosslink the polymer.

### D. Assembly of PDMS Projection Array Substrates in 24 Well Microplates

Once the PDMS projection arrays were made, they were subject to surface oxidation using O₂ plasma cleaning for 30 seconds at pressure of 500 mTorr, and put onto the bottom of each well of a 24-well microplate. Sufficient adherence between the projections of the arrays and the well of the microplate was obtained by pressing the arrays of projections against the surface of the well. Afterwards, each well was filled with 75% ethanol twice, each 30 sec, followed by washing with PBS buffer and drying. For some experiments, a PBS buffered Collagen I solution (200 μl) was added into each well, and incubated for 45 min. After aspiration of the Collagen I solution, the surface of each well was air-dried.

### E. Cell Culture

HepG2C3A (CRL-1074) human hepatoblastoma cell line was purchased from American Type Culture Collection and cultured in MEM Eagle medium containing 1 mM sodium pyruvate, 10% (v/v) fetal bovine serum (FBS), and 2 mM L-glutamine. All cell cultivation, HepG2C3A cells were seeded in 24-well plates. The cells were cultured under standard conditions: a humidified atmosphere of 5% CO₂ and 95% air at 37° C. with daily medium changes. The cells were seeded at a density of 20,000, 40,000 or 80,000 density on each PDMS substrate. Duplicates for each condition were examined. Collagen I microplates from BD Biosciences were used as control.

Both immortalized liver cell line F2N-4 and primary liver cells were purchased from MultiCell Inc. and cultured in plating medium for one day, and substituted with maintenance medium with daily exchanges, using the protocol as recommended by the supplier.

Cryopreserved primary hepatocyte cells were purchased from XenoTech (H1500.H15A+Lot No. 770). Cells were thawed and purified using Xenotech Hepatocyte isolation kit (Cat#: K2000) according to the manufacturer's instructions. Cells (50,000/well) were plated in collagen I coated 96-well plate (BD Bioscience, Cat# 354407) or uncoated PDMS microprojection array substrates using Galactose-free MFE Plating Medium (Corning Inc.) containing 10% FBS on Day 1. The medium was changed to MFE Maintenance Medium containing 10% FBS with 0.25 mg/ml MATRIGEL™ (BD Bioscience, Cat#356234 or 354510) on Day 2. Cells were incubated at 37° C. with 5% CO2 from Day 1 to Day 8.

### F. Immunostaining and Fluorescence Imaging

To perform F-actin staining, the manufacturer recommended protocol was largely used. Briefly, cells were fixed using 3.7% formaldehyde, permeabilized for 5 min in 0.1% Triton X-100 in 1% bovine serum albumin (BSA), blocked in 10% bovine serum albumin at specified temperature for a given period, incubated with TR-phalloidin (1 μg/ml) for 1 hr and then wash 3 times with phosphate buffer saline (PBS) before imaging.

For Live/Dead cell staining, the Live/Dead cell staining reagent kits from Molecular Probes (Eugene, Oreg.) were used with the manufacturer's recommended protocol. All microscopic images were obtained using Zeiss microscope.

### G. MTS Assays

Hepatocyte proliferation was examined using an MTS assay. 3-(4,5-dimethylthiazol-2-yl)-5-(3-carboxy-methoxyphenyl)-2-(4-sulfophenyl)-2H-tetrazolium inner salt). (MTS) and phenazine methosulfate (PMS) were obtained from Promega (Madison, Wis.) and Sigma-Aldrich Chimie, respectively. MTS (2 mg/mL; pH 6.5) was dissolved in PBS and filter sterilized. A 3 mM PMS solution was also prepared (in PBS) and filter sterilized. These solutions were stored at −20° C. in light-protected containers. To enhance the cellular reduction of MTS, PMS was added to MTS immediately before use (MTS-PMS ratio: 1:20). A portion of the mixture (150 μL) was added to each well. After cell culture for 24 hours, 100 microliters of the supernatant was diluted in 1 milliliter deionized water. The optical density was measured at 490 nm by means of spectrophotometry. Cell growth was analyzed by means of MTS assay after 24 hours of culture. Cell proliferation also was analyzed with a hemocytometer and a cell counter (Beckman Coulter, Fullerton, Calif.).

### H. CYP3A4 Induction Assays

The Promega kit (Invitrogen, Corporation, Carlsbad, Calif.) was used for drug effect studies. Briefly, cells were cultured for specific time on PDMS substrates with microprojection arrays. After 3 days continuous drug (rifampin) induction, the substrates were washed with media/PBS twice. Add 200 μl luminogenic substrate (Luciferin-PFBE, 1:40 dilution in media) to all wells and incubate at 37° C. for 3-4 hours. 50 microliters of the reaction from the well were transferred and 50 microliters of Luciferin detection reagent were added and, incubated for another 20 minutes at room temperature. Luminescence readings were taken using a luminometer to check the results.

### I. Culture and Gene Expression Analysis of Primary Liver Cells

For cryopreserved primary liver cells, the cells as received were thawed to room temperature and lysed directly without any further culture in vitro. For primary liver cells cultured on the PDMS microprojection substrates, the cells were cultured on different PDMS substrates directly, overlaid in solution with MATRIGEL™ at the 2^{nd} day and continued with further culture without any serum for 6 days. Afterwards, the hepatocytes cultured were harvested and total RNA were extracted using Qiagen RNeasy Mini kit (Cat#74104) on column DNase digestion (Cat#79254). RNA concentration of each sample was quantified with Quant-iT™ RiboGreen® RNA Assay Kit (Invitrogen, Cat#R11490) and stored at −80° C. until PCR-array experiments. Array plates (Human Cancer Drug Resistance & Metabolism PCR Array, Cat#PAHS-004, SABioscience, Frederick, Md.) were prepared following SA Bioscience manual (Part#1022A). 250 ng total RNA was used per 96-well array plate. The PCR-Array was performed on an ABI-7300 with 96-well standard block using software SDS1.3. PCR conditions were set up as suggested in the user manual (Part#1022A). Data was analyzed using SA Bioscience online analysis tool.

### II. Hepatocyte Cells Cultured on Oxidized and Collagen I Coated PDMS Projection Array Substrates

Due to the importance of reestablishing membrane polarity in maintaining functions of hepatocytes, the ability of the projection array substrates for prompting cell attachment and growth, and maintaining the membrane polarity of cultured hepatocytes was examined. For purposes of illustration, FIG. 10 depicting the polarity of schematic hepatocytes 800 in vivo is provided. In the liver, the basal lateral cell membrane 810 of the hepatocytes 800 is exposed to the liver sinusoid, or the blood supply, as well as the narrow intercellular space between adjacent hepatocytes. The apical domain of the cell membrane 820 is exposed to the tube or space between liver cells that collects bile from the cell (i.e., the bile canaliculus). Sinusoidal cells 900 are also depicted in FIG. 10.

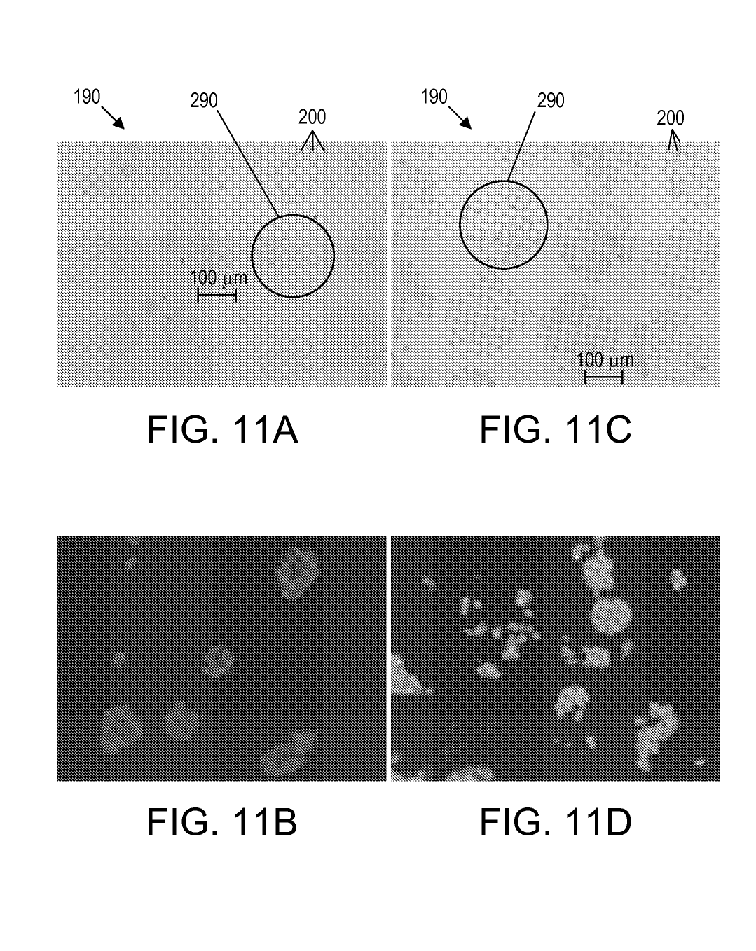

FIG. 11 shows microscopic images of hepatocyte hepG2C3A cells cultured on two distinct types of oxidized PDMS projection array substrates. The projection microarray 190 (see for example 190 in FIG. 7A) substrates contain microarrays 290 of projections (see 290 in FIG. 7A). In FIGS. 11A and 11B, each projection 200 in a projection microarray 290 has a diameter of 5 microns and a height of 5 microns, and the gap distance (d) (see FIG. 9) between the nearest projections is 10 microns. In FIGS. 11C and 11D, each projection in a projection microarray 290 has a diameter of 15 microns and a height of 5 microns, and the gap distance (d) between the nearest projections is 25 microns. The seeding numbers for both types of projection array substrates were 40,000 cells per well in a 24 well microplate. After 1 day culture and stained with the Live/Dead staining reagent, the cells on the substrates were examined with both phase contrast light microscopic imaging (FIGS. 11A and 11C), as well as with fluorescence imaging (FIGS. 11B and 11D). As shown in these images, the cells were preferably attached on the projection microarray 290 area regardless of the projection spacing. The fluorescence images indicate that all (or nearly all) the hepatocytes are alive once cultured onto these substrates, as evidenced by which most of cells appear green in fluorescence (an indicator of alive cells), and little is red in fluorescence (an indicator of dead cells).

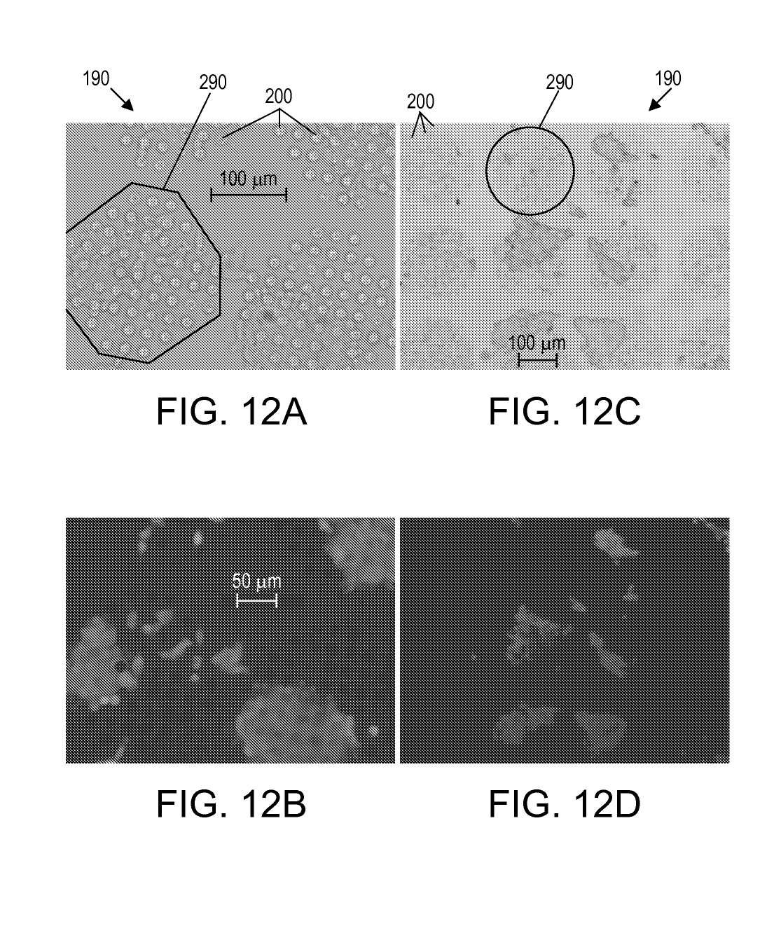

FIG. 12 shows microscopic images of hepatocyte hepG2C3A cells cultured on two distinct types of oxidized PDMS projection substrates. Here the images were obtained after 4 day cultures. In FIGS. 12A and 12B, each projection 200 in a projection microarray 290 has a diameter of 15 microns and a height of 5 microns, and the gap distance (d) between the nearest projections is 25 microns. In FIGS. 12C and 12D, each projection 200 in a projection microarray 290 has a diameter of 5 microns and a height of 5 microns, and the gap distance (d) between the nearest projections is 10 microns. In this experiment, the seeding numbers for both types of projection array substrates were 20,000 cells per well in a 24 well microplate. After 4 days of culture cells were stained with the Live/Dead staining reagent, the cells on the substrates were examined with both phase contrast light microscopic imaging (FIGS. 12A and 12C), as well as with fluorescence imaging (FIGS. 12B and 12D). Once again as shown in these images, the cells were preferably attached onto the projection array area. While not evident in black and white reproductions no red fluorescent staining is evident. The lacking of any red fluorescence suggested that the hepatocytes are alive once cultured onto these substrates. On the projection array substrate having the projections with the shorter gaps (FIGS. 12C and 12D), the cells tended to form 3-dimensional clusters, indicating that the hepG2C3A cells were more physiologically viable in the smaller spaced projection microarray.

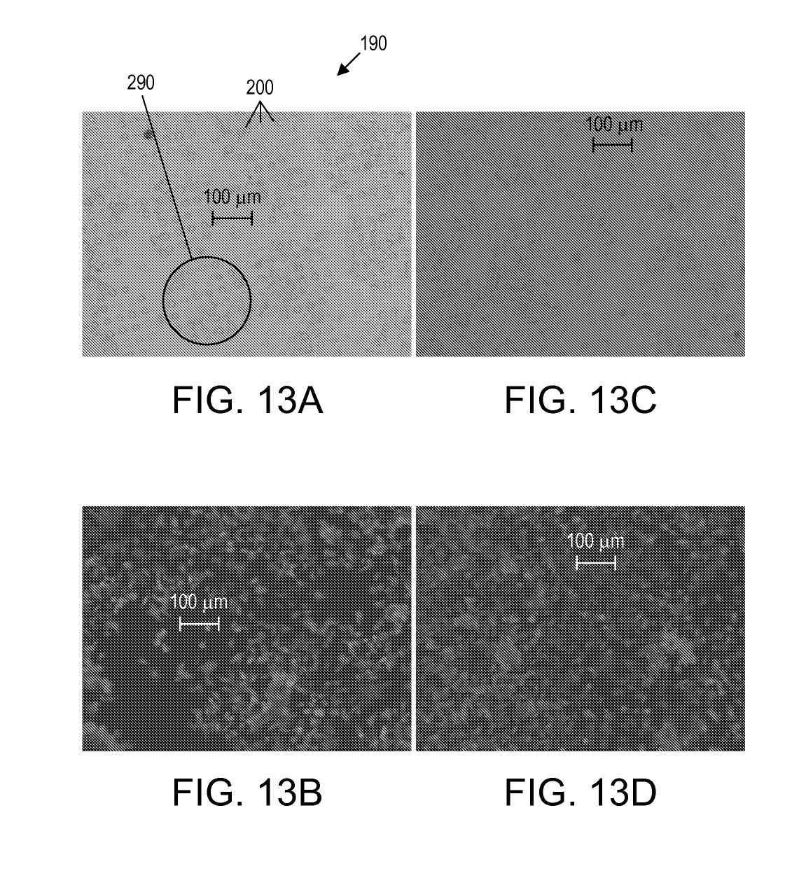

FIG. 13 shows microscopic images of hepatocyte hepG2C3A cells cultured on two distinct types of collagen I-coated PDMS projection substrates. Here the images were obtained after 4 day cultures. In FIGS. 13A and 13B, each projection 200 in a projection microarray 290 has a diameter of 10 microns and a height of 5 microns, and the gap distance between the nearest projections is 20 microns. In FIGS. 13C and 13D, each projection in a projection array has a diameter of 5 microns and a height of 5 microns, and the gap distance between the nearest projections is 5 microns. In this experiment, the seeding numbers for both types of projection array substrates were 20,000 cells per well in a 24 well microplate. After 4 days culture and stained with the Live/Dead staining reagent, the cells on the substrates were examined with both phase contrast light microscopic imaging (FIGS. 13A and 13C), as well as with fluorescence imaging (FIGS. 13B and 13D). Results showed that again almost all cells were alive on these substrates, and interestingly on the collagen I coated projection substrates the cells tended to grow into a monolayer.

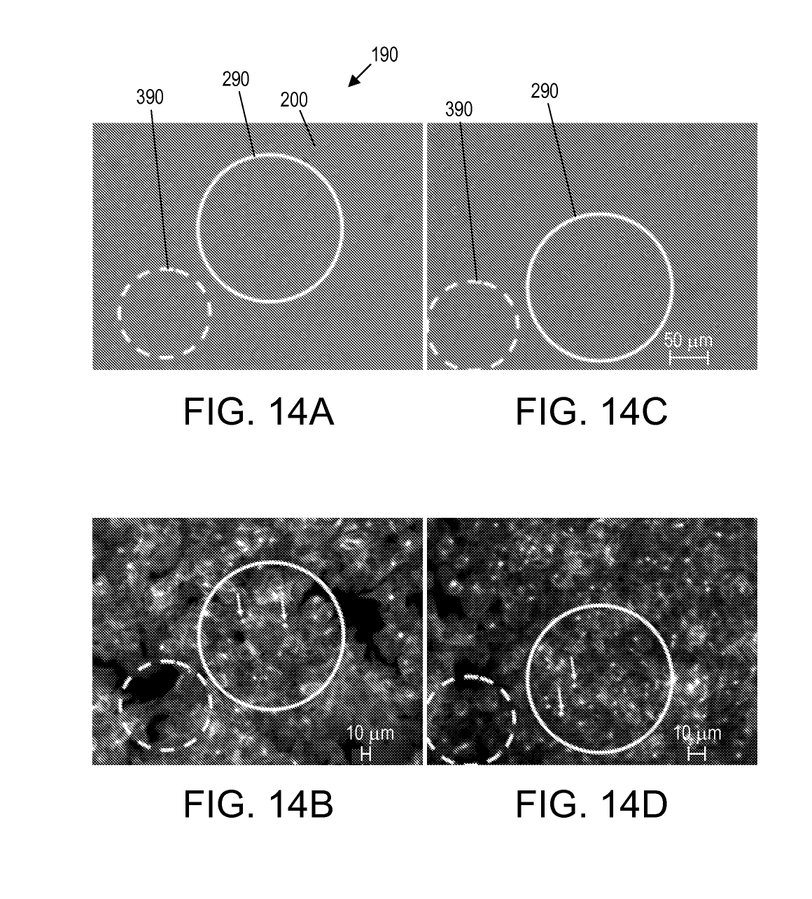

FIG. 14 shows microscopic images of hepatocyte hepG2C3A cells cultured on two distinct types of collagen I-coated PDMS projection substrates. Here the images were obtained after 4 day cultures. In FIGS. 14A and 14B, each projection 200 in a projection microarray 290 has a diameter of 10 microns and a height of 5 microns, and the gap distance (d) between the nearest projections 200 is 20 microns. In FIGS. 14C and 14D, each projection 200 in a projection microarray 290 has a diameter of 10 microns and a height of 5 microns, and the gap distance (d) between the nearest projections 200 is 25 microns. In this experiment, the seeding numbers for both types of projection array substrates were 40,000 cells per well in a 24 well microplate. The cells were continuously cultured for 4 days culture, followed by fixation and staining with Texas Red-x-phallodin, and finally examined with both phase contrast light microscopic imaging (FIGS. 14A and 14C), as well as with fluorescence imaging (FIGS. 14B and 14D). As shown in the images, the cells tended to form a monolayer. Interestingly, the cells exhibited unique polarity, as revealed by the actin staining patterns. For cells located within the projection area 290 (as indicated by the solid circles), the actin filaments primarily concentrated on one side of each cell (as indicated by the white arrows), suggesting the formation of the bile canaliculus—a marker for in vivo-like polarity of hepatocyte cells. The striking in vivo-like polarity of cultured hepatocytes on these projection array substrates represents the first ever experimentation evidence that in vitro hepatocyte cell culture can lead to in vivo-like cell morphology under non-sandwich and monolayer culture conditions. The membrane polarity is an important indicator for the functions of in vitro cultured hepatocytes. On the other hand, on the area between the microprojection microarrays as indicated by the dotted line circles (390), cells tend to give rise to little or no concentrated actin filament staining patterns, indicating that cells on these areas did not restore their polarity.

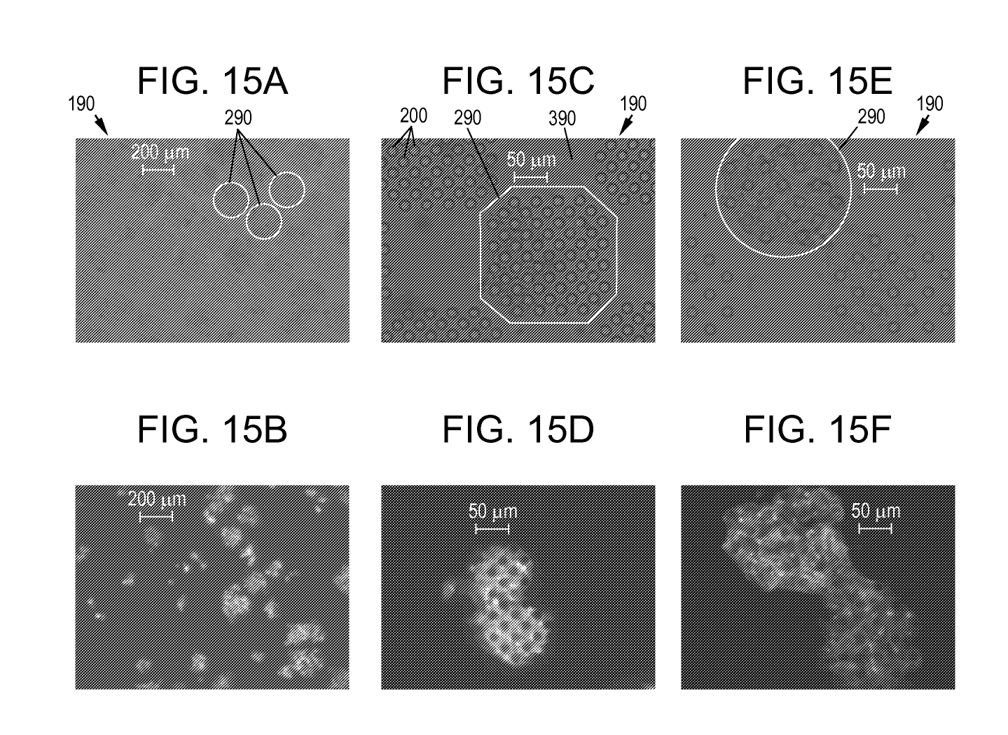

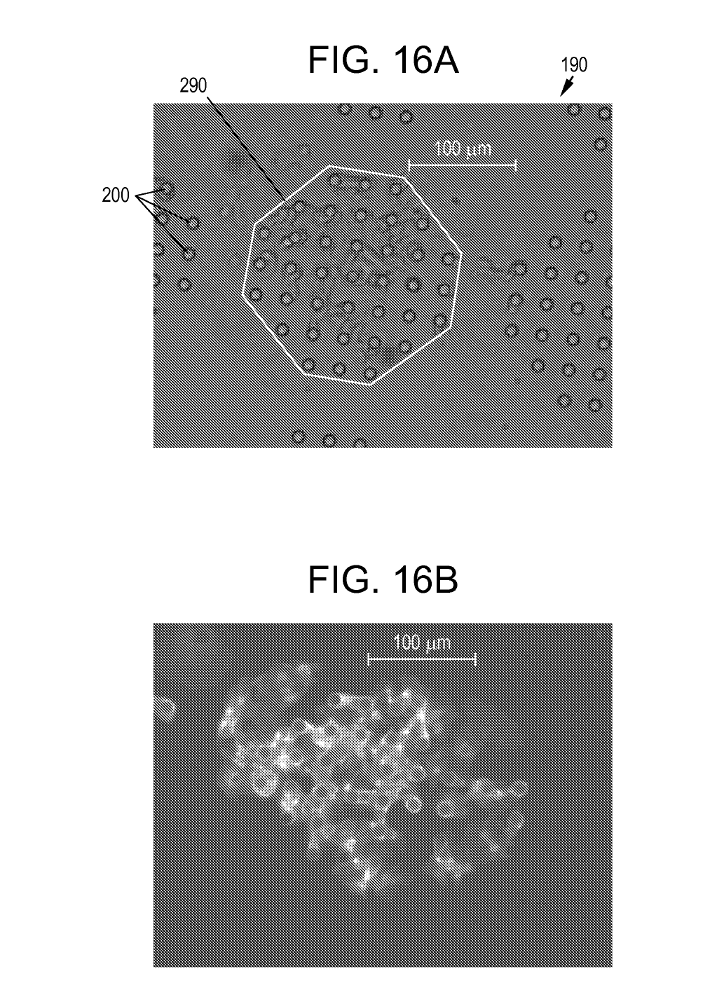

FIG. 15 shows microscopic images of hepatocyte hepG2C3A cells cultured on two distinct types of oxidized coated PDMS projection substrates. Here the images were obtained after 5 day cultures. In FIG. 15A-D, each projection 200 in a projection microarray 290 has a diameter of 15 microns and a height of 5 microns, and the gap distance (d) between nearest projections is 20 microns. In FIGS. 15E and 15F, each projection 200 in a projection microarray 290 has a diameter of 15 microns and a height of 5 microns, and the gap distance (d) between the nearest projections is 25 microns. In this experiment, the seeding numbers for both types of projection array substrates were 20,000 cells per well in a 24 well microplate. The cells were continuously cultured for 5 days culture, followed by fixation and staining with Texas Red-x-phallodin, and finally examined with both phase contrast light microscopic imaging (FIGS. 15A and 15C and 15E), as well as with fluorescence imaging (FIGS. 15B and 15D and 15F). As shown in the images, the cells tend to form clusters on the projection area only. Interestingly, the cells exhibited unique polarity, as revealed by the actin staining patterns shown in FIGS. 15B, 15D and 15F. The actin filaments primarily concentrated on one side of each cell, suggesting the formation of the bile canaliculus—a marker for in vivo-like polarity of hepatocyte cells.

FIG. 16 shows microscopic images of hepatocyte hepG2C3A cells cultured on a collagen I-coated PDMS projection substrates. Here the images were obtained after 5 day cultures. Here, each projection 200 in a projection microarray 290 has a diameter of 10 microns and a height of 5 microns, and the gap distance between the nearest projections is 20 microns. In this experiment, the seeding numbers for both types of projection array substrates were 20,000 cells per well in a 24 well microplate. The cells were continuously cultured for 5 days culture, followed by fixation and staining with Texas Red-x-phallodin, and finally examined with both phase contrast light microscopic imaging (FIG. 16A), as well as with fluorescence imaging (FIG. 16B). As shown in the images, the cells again tend to form monolayer clusters, and mainly located at the projection area. Similarly, the cells exhibited unique polarity, as revealed by the actin staining patterns shown in FIG. 16B. The actin filaments primarily concentrated on one side of each cell, suggesting the formation of the bile canaliculus—a marker for in vivo-like polarity of hepatocyte cells.

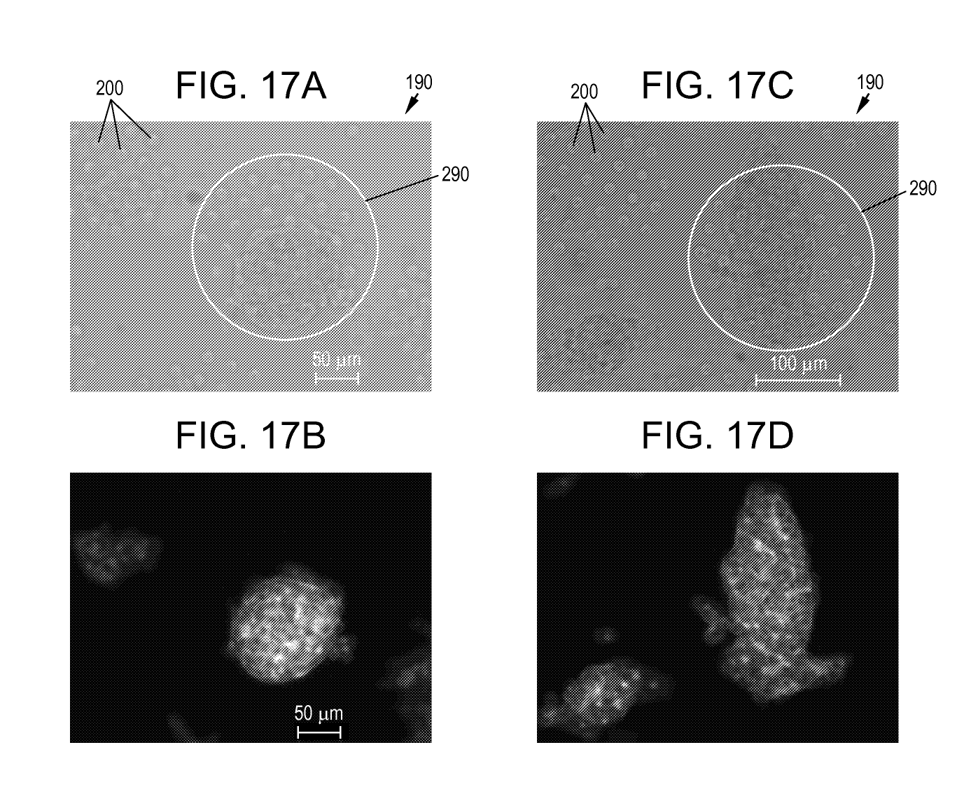

FIG. 17 shows microscopic images of hepatocyte hepG2C3A cells cultured on oxidized PDMS projection substrates. Here the images were obtained after 7 day cultures. Here, each projection 200 in a projection microarray 290 has a diameter of 10 microns and a height of 5 microns, and the gap distance between the nearest projections is 20 microns. In this experiment, the seeding numbers for both types of projection array substrates were 80,000 cells per well in a 24 well microplate. The cells were continuously cultured for 7 days culture, followed by fixation and staining with Texas Red-x-phallodin, and finally examined with both phase contrast light microscopic imaging (FIGS. 17A and 17C), as well as with fluorescence imaging (FIGS. 17B and 17D). As shown in the images, the cells again tend to form three dimensional clusters, and mainly located at the projection area. Similarly, the cells exhibited unique polarity, as revealed by the actin staining patterns shown in FIGS. 17B and 17D. The actin filaments primarily concentrated on one side of each cell, suggesting the formation of the bile canaliculus—a marker for in vivo-like polarity of hepatocyte cells.

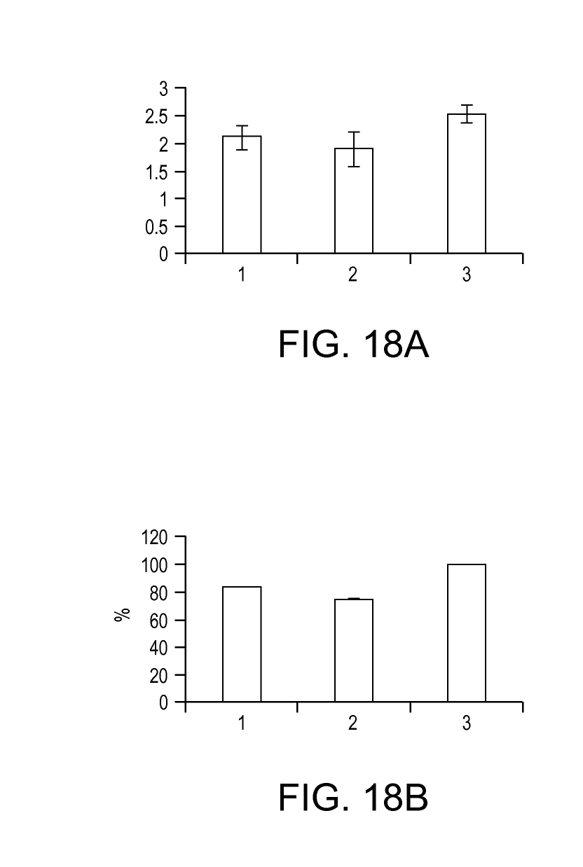

FIG. 18 shows results of hepatocyte hepG2C3A cell proliferation on three types of substrates: collagen I coated PDMS projection substrate (1), oxidized PDMS projection substrate uncoated (2), and collagen I coated tissue culture treated polystyrene substrate (3). Here each projection in a projection array has a diameter of 10 microns and a height of 5 microns, and the gap distance between the nearest projections is 20 microns. In this experiment, the seeding numbers for these substrates were 100,000 cells per well in a 24 well microplate. After one day, the cells were examined with MTS assays. Results showed that the hepatocytes on both projection microarray substrates led to slightly smaller readings than those on the collagen I coated TCT surfaces, suggesting that the cell proliferation and viability on these projection microarray substrates are slightly slower than that on collagen I coated TCT surfaces.

### III. Co-Culture of Hepatocyte Cells and Helper Cells on Oxidized PDMS Projection Array Substrates

NIH3T3 fibroblast cells were co-cultured with hepG2C3A cells on oxidized PDMS projection array substrates prepared as described above.

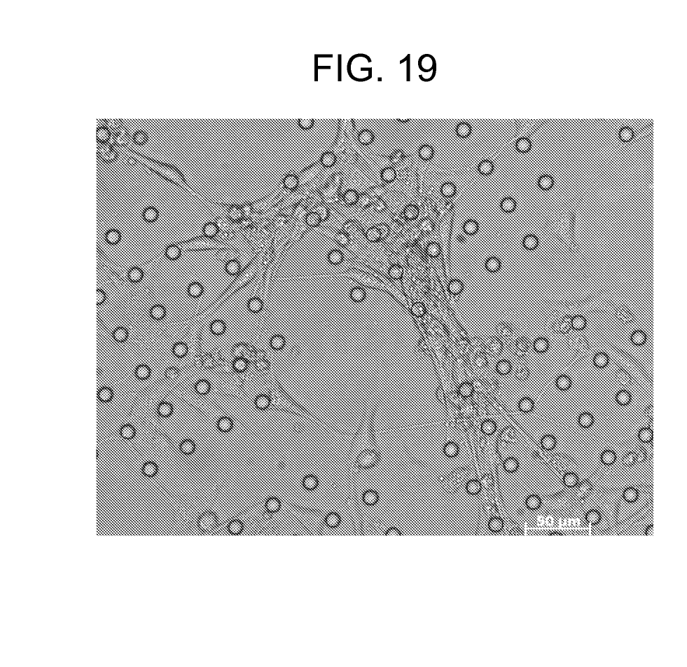

FIG. 19 shows a light microscopic image of NIH3T3 fibroblast cells in the absence of hepG2C3A cells, on oxidized PDMS microprojection array substrate is shown. The NIH 3T3 cells were grown on plasma treated PDMS microprojection substrate which has arrays of microprojections of 10 micrometers in diameter and 25 micrometers in the gap distance between the two nearest projections. The image shown in FIG. 19 was taken after 6 days cell culture. The initial seeding density was 40 k/well in a 24 well microplate. It is worth noting that the cells also tend to grow within the projection microarray areas.

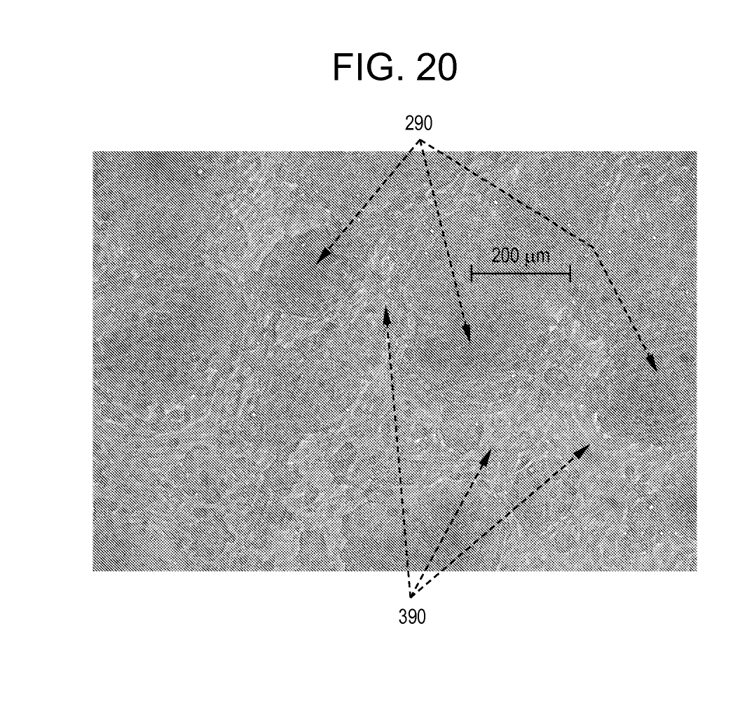

FIG. 20 is a light microscopic image of co-culture of NIH3T3 fibroblast cells and hepG2C3A cells on oxidized PDMS microprojection array substrate having arrays of microprojection of 10 μm in diameter and 20 μm in the gap distance between the two nearest projections. Here NIH 3T3 cells of 40,000 cells per well in a 24 well microplate in the DMEM (Dulbecco's Modified Eagle Medium) medium were seeded and pre-cultured on the substrate for 1 day, followed by an overlay with HepG3C3A cells of 40 k cells per well in the MEME (Minimum Essential Media Eagle) medium. The image was taken after 9 days of co-culture. Results showed that the hepatocyte cells form three dimensional clusters within the microprojection array area, whereas NIH3T3 cells predominantly located between the HepG2C3A cell clusters and on the flat area between the microprojection arrays. It is interesting to note that although NIH3T3 cells primarily locate between the HepG2C3A cell clusters and on the flat area between the microprojection arrays, the NIH3T3 cells could adhere on the major surface within the microprojection arrays (see FIG. 19) and form the basal layer of cells under the HepG2C3A cells or cell clusters.

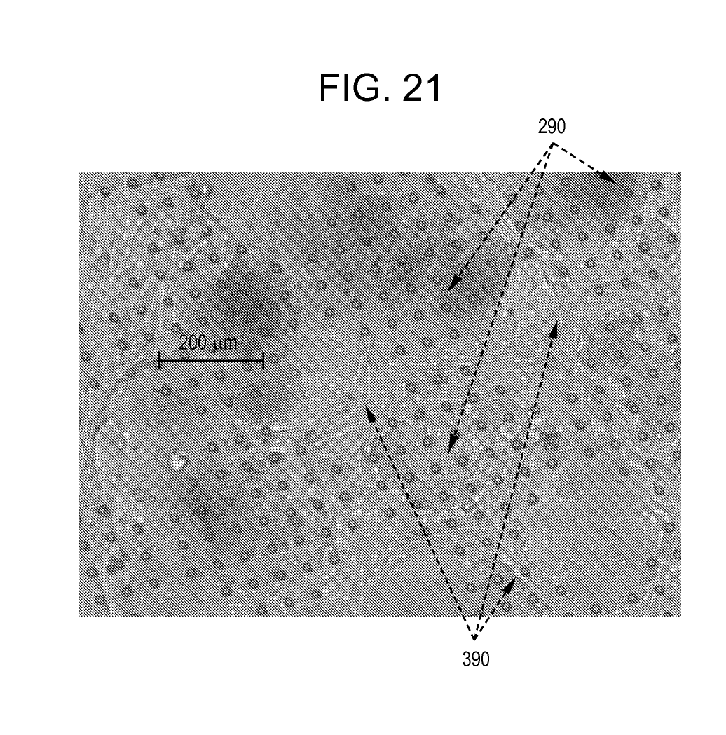

FIG. 21 is a light microscopic image of co-culture of NIH3T3 fibroblast cells and hepG2C3A cells on oxidized PDMS microprojection array substrate having arrays of microprojections 200 of 10 μm in diameter and 20 μm in the gap distance (d) between the two nearest projections. Here HepG2C3A cells of 40,000 cells per well in a 24 well microplate in the MEME medium were seeded and pre-cultured onto the substrate for one week, followed by an overlay with NIH3T3 cells of 40 k cells per well. The image was taken after 9 days co-culture. Results showed that the C3A cells form 3-D clusters within the microprojection array area 290, whereas NIH3T3 cells predominantly located between the c3A clusters 390 and on the flat area between the microprojection arrays.

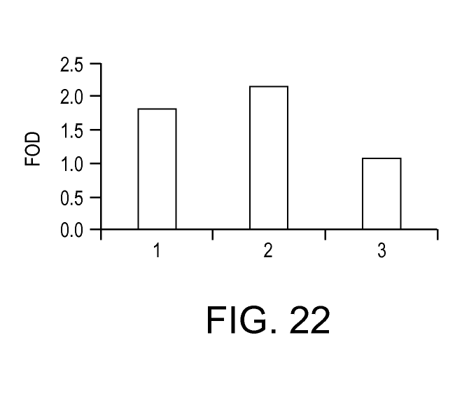

FIG. 22 is a graph of rifampin-induced CYP3A4 enzyme activity of HepG2C3A cells co-cultured with NIH3T3 cells on the oxidized PDMS microprojection substrates, as shown in FIG. 20 and FIG. 21. The fold of induction (FOD, Y axis) of CYP3A4 by rifampin was obtained after 72 hours continuous drug induction, and measured using a Promega CYP Kit. The cell numbers were normalized on three culture conditions: C3A cells subsequently co-cultured with 3T3 cells (1); NIH3T3 cells subsequently co-cultured with C3A cells (2); and C3A cells cultured on BD Biosciences' Collagen coated 24 well microplate (3). Results showed that rifampin induction increases the CYP3A4 function by almost 100% on both co-culture conditions, but did not cause any increase on the collagen-coated surfaces.

### IV. Long Term Culturing of Hepatocyte Cells on Oxidized and Collagen I Coated PDMS Projection Array Substrates

Conventional 2-D sandwich or 3-D MATRIGEL™ culture of hepatocytes is generally limited to short-term culture (e.g., 1 week or so). After long-term culture, these cultured hepatocytes can loss some of their viability or their metabolic functions. Projection array substrates as described herein support the long-term culture of hepatocyte cells.

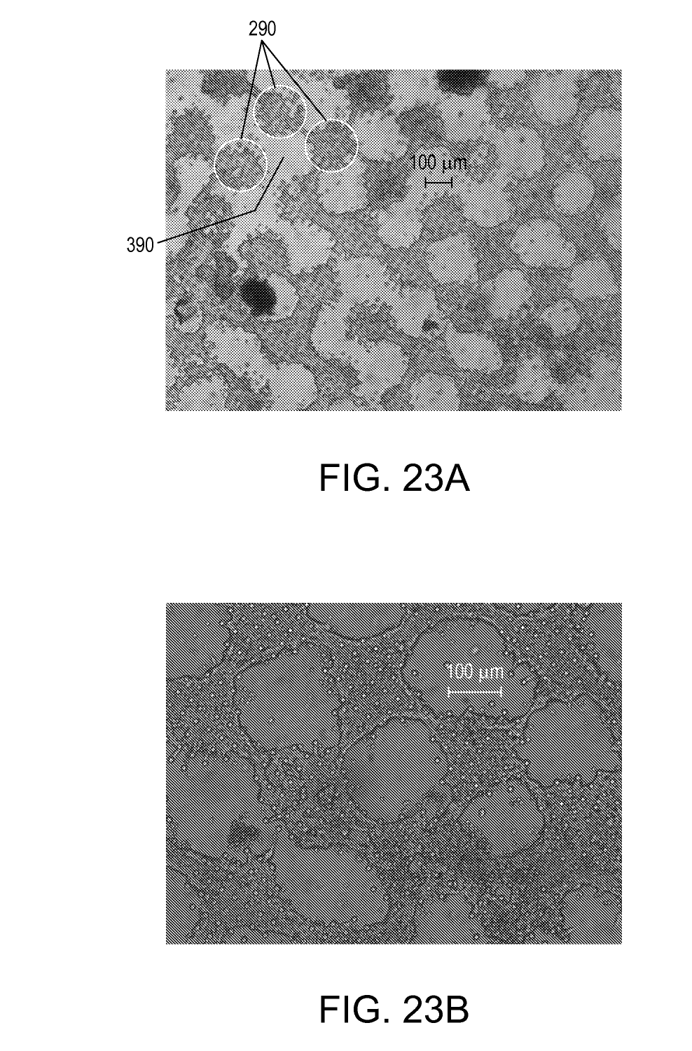

FIG. 23 shows light microscopic images of hepatocyte hepG2C3A cells cultured on an oxidized PDMS projection array substrate. The projection array substrates contain arrays 290 of projections 200. In FIGS. 23A and 23B, each projection 200 in a projection microarray 290 has a diameter of 5 microns and a height of 10 microns, and the gap distance (d) between the nearest projections is 20 microns. The seeding numbers for the projection array substrates was 40,000 cells per well in a 24 well microplate. After 28 days culture, the cells on the substrates were directly examined with phase contrast light microscopic imaging. As shown in these images, the cells were preferably attached onto the projection array area 290 and form 3-dimensional clusters. Interestingly, after such long-term culture, the C3A cell clusters between the two nearby microprojection arrays can communicate each other (FIG. 23B).

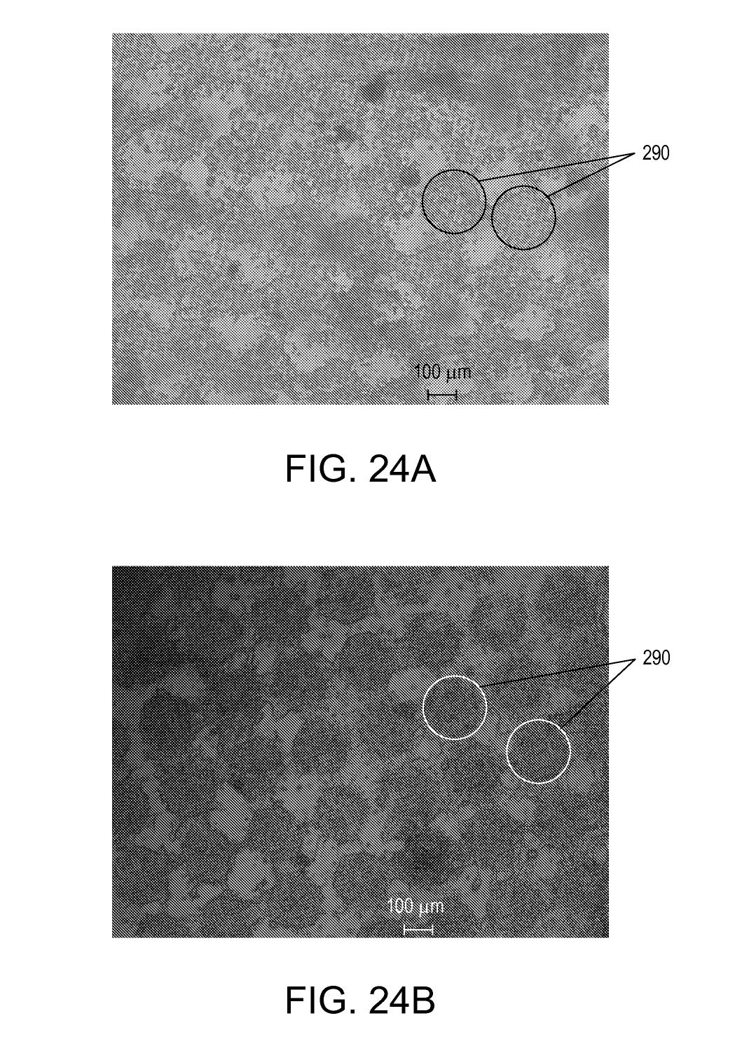

FIG. 24 shows light microscopic images of hepatocyte hepG2C3A cells cultured on a collagen I coated oxidized PDMS projection array substrate. The projection array substrates contain arrays of projections. In FIG. 24A, each projection 200 in a projection microarray 290 has a diameter of 5 microns and a height of 5 microns, and the gap distance (d) between the nearest projections is 15 microns. In FIG. 24B, each projection in a projection array has a diameter of 5 microns and a height of 5 microns, and the gap distance between the nearest projections is 10 microns. The seeding numbers for the two projection array substrates were 40,000 cells per well in a 24 well microplate. After 28 days culture, the cells on the substrates were directly examined with phase contrast light microscopic imaging. As shown in these images, the cells were preferably attached onto both projection array area and form 3-dimensional clusters. Interestingly, after such long-term culture, the HepG2C3A cell clusters between the two nearby microprojection arrays also can communicate each other.

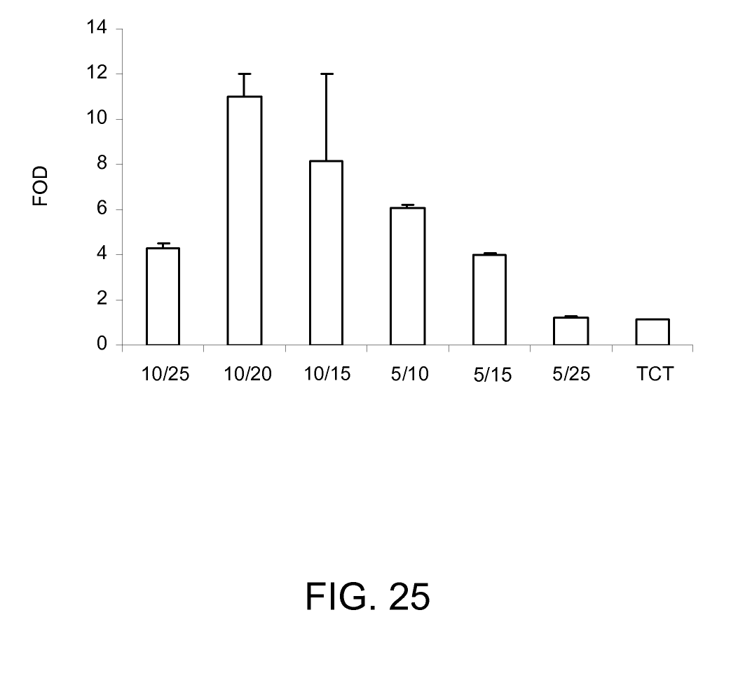

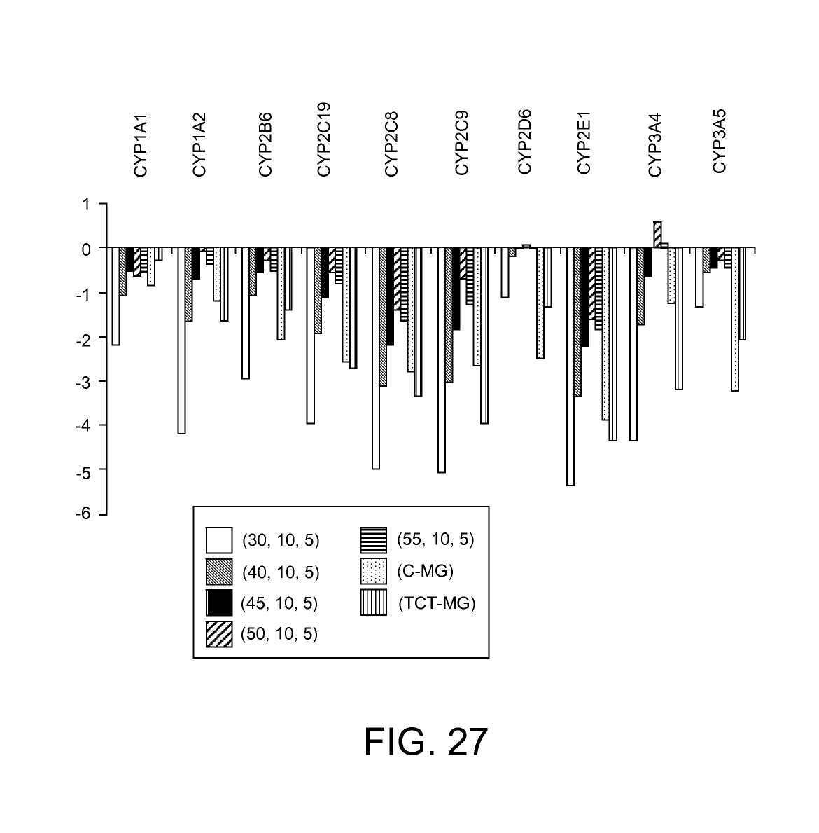

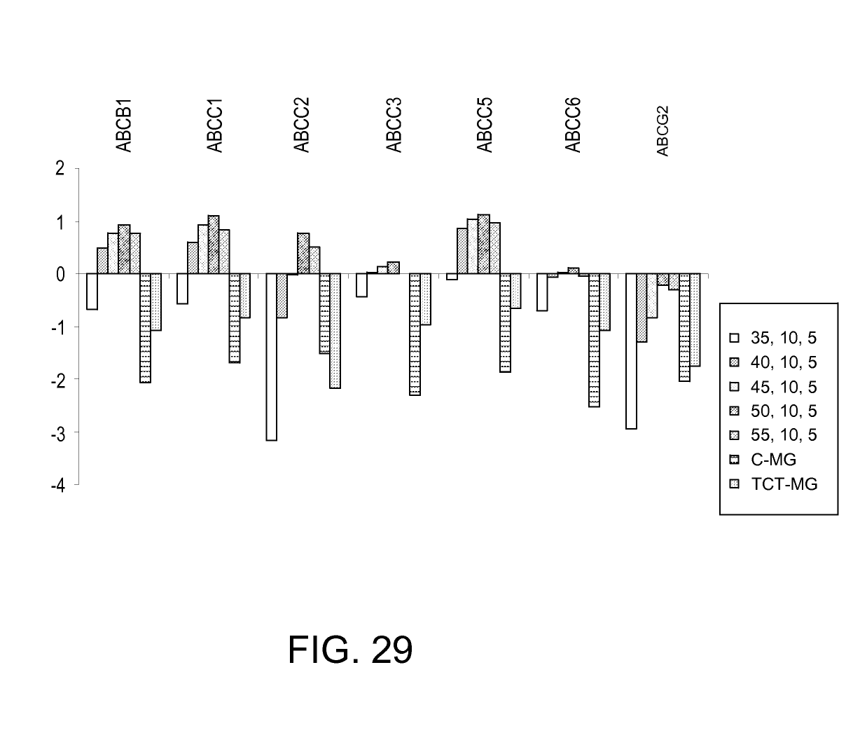

FIG. 25 is a graph of rifamipin-induced CYP3A4 enzyme activity of HepG2C3A cells cultured onto the oxidized PDMS microprojection substrates, as exampled in FIG. 23. Here different microprojection substrates were used. In comparison, cells on the TCT surface were used as a negative control. After 28 days culture, the CYP3A4 induction, i.e., the fold of induction (FOD) of CYP3A4 by rifampin, was obtained after 72 hours continuous drug induction, and measured using a Promega CYP Kit. The cell numbers were normalized on all culture conditions. The parameters of the microprojection array substrate are listed on the X-axis as x/y, wherein x refers to the height of the microprojection in each array, and y refers to the gap distance (d) between the two nearest microprojections, both in micrometers. Results showed that the most influential parameter is the gap distance between the neighboring microprojections, and the optimal distance is between 10 and 25 microns, or close to the two fold of the size of a single HepG2C3A cell for these cells under these conditions. Such gap distance dependent maximal CYP3A4 induction was found to be true to either F2N4 cells or primary liver cells (FIGS. 27 and 29; and data not shown). These results suggest that the hepatocyte cells can survive long term culture and have very strong function expression under drug induction. Notably, the HePG3C3A cells on the collagen I coated PDMS substrate tend to form monolayers after short term culture (˜5 days). However, after 4 weeks of cell culture, we found that they also form networks following the arrangement of the microprojection arrays, and cells that previously stayed on the non-microprojection array region now disappeared. One possibility is that cells in non-microprojection array region can not survive long time culture but in the microprojection array region, cells grow very healthily, which was proved by the formation of the high quality networks, as well as the drug induction experimental results.

The results of the co-culture studies revealed several key findings. First, microprojection array substrates support long term cell growth. Co-cultured HepG2C3A cells preferentially stay within areas defined by the projection arrays, while NIH 3T3 cells preferentially stay in the spaces between arrays. Such an arrangement mirrors in vivo arrangements where hepatocytes tend to group together. Further evidence that co-culturing on the microprojection array substrates emulates in vivo function is shown by the results indicating that co-culture of NIH 3T3 with HepG2C3A can increase C3A4 P450 expression. In addition, microprojection array substrates were shown to control cell morphology and cell-cell communications. Notably, compared to cells grow on 2-dimensional substrates, which lose their polarity rapidly, cells grow on the microprojection array substrates restore their membrane polarity and exhibit enhanced function expression.

### V. Gene Expression Analysis of Primary Liver Cell Cultured on Different PDMS Projection Array Substrates

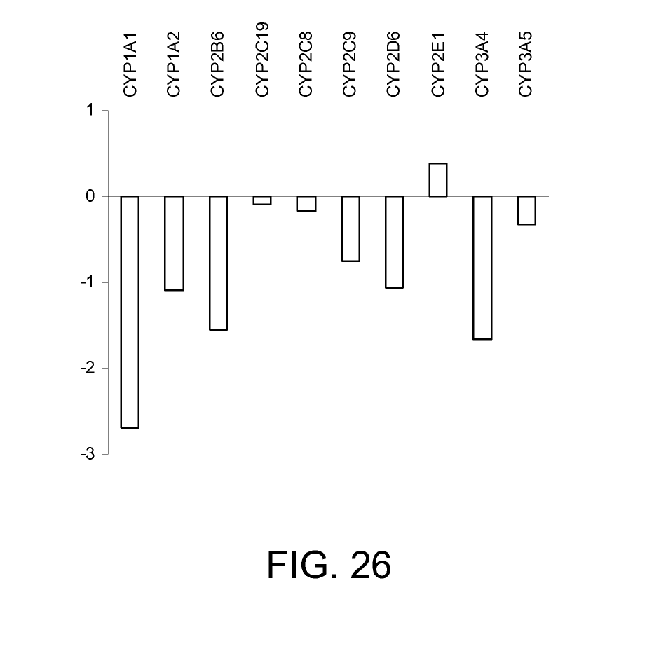

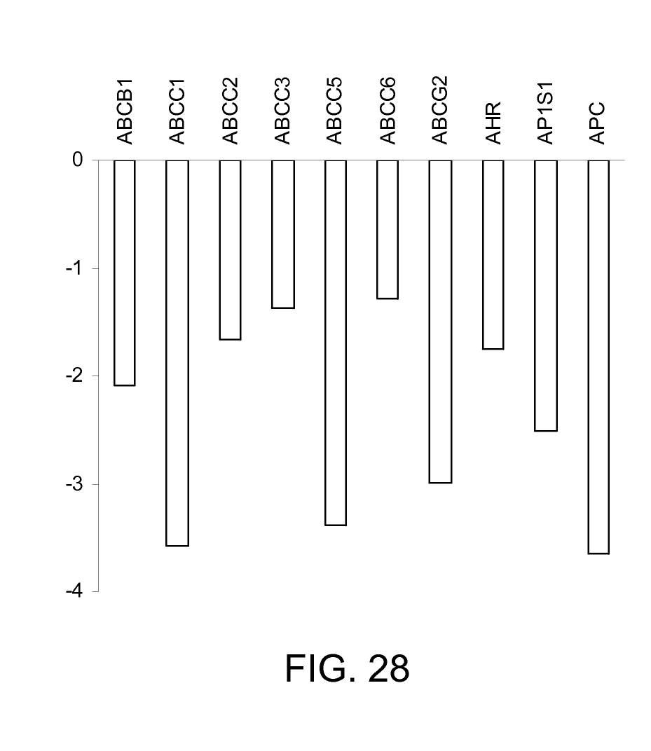

Gene expression analysis has become popular in assessing the function of in vitro cultured primary liver cells. We used SABiosciences' Human Cancer Drug Resistance & Metabolism PCR Array to systematically assess the expression of two sets of important genes in hepatocytes function: 10 CYP genes and 10 transporter genes. For comparison, the cryopreserved primary hepatocytes were also analyzed. FIG. 26 showed log of fold change in basal mRNA expression of cryopreserved hepatocytes relative to GADPH control gene expression for 10 CYP genes without any in-vitro culture after normalized to internal control gene GADPH. Results showed that compared to moderate expression of the control GADPH gene, all CYPs were expressed in this cryopreserved hepatocytes but with a relatively lower expression; and different CYP genes gave rise to different expression level and CYP2E1 had the highest expression. Similarly, the cryopreserved hepatocytes also express all 10 transporter genes but at much lower levels, as shown in FIG. 28 after normalized to the internal control gene GADPH.

FIG. 27 shows the log of fold change in gene expression of 10 CYP genes (CYP1A1, CYP1A2, CYP2B6, CYP2C19, CYP2C8, CYP2C9, CYP2D6, CYP2E1, CYP3A4 AND CYP3A5) in hepatocytes cultured on 5 different microprojection substrates relative to cryopreserved hepatocytes (results shown in FIG. 26), in comparison with two controls: cells sandwiched on collagen I-coated TCT and MATRIGEL™, and cells sandwiched on uncoated TCT and MATRIGEL™. The PDMS projection microarray substrates were defined as (X, Y, Z) wherein X is the gap distance (d) in micrometer between the two nearby projections, Y is the diameter in micrometer of the projection, while Z is the height in micrometer of the projection. The projection microarray is hexagonal. Results showed that after 7 days in vitro culture on different projection microarray substrates using a modified MATRIGEL™ Overlay culture, we found that all CYP genes were still detectable in the hepatocytes cultured on all projection microarray substrates; and the CYP gene expression gave rise to a microprojection gap distance (d) dependence, and the substrate having a (d) of 50 micrometers (i.e., closely to be the twice in size of a primary hepatocyte cell) gave rise to the highest expression of almost all CYP genes. Except for the projection microarray substrate having the smallest gap distance (d) (35 micrometers), all PDMS projection microarray substrates gave rise to higher CYP gene expression than the two controls.

FIG. 29 shows log of fold change in basal mRNA expression (ABCB1, ABCC1, ABCC2, ABCC5, ABCC6, ABCG2, AHR, AP1S1 and APC) of 10 transporter genes in cultured hepatocytes on the projection microarray substrates relative to cryopreserved hepatocytes, in comparison with those on the two control substrates. Results showed that the hepatocytes cultured on all projection microarray substrates except for the smallest gap distance (d) substrate (i.e., the (35, 10, 5) substrate) gave rise to higher expression of almost all transporter genes, than the cryopreserved hepatocytes as well as hepatocytes cultured on the two control substrates. These results suggest that given appropriate design of the projection microarray substrates (particularly the gap distance), the primary hepatocytes cultured maintain high level of expression of CYP genes, as well as gain high expression of transporter genes; and such high expression of the two classes of drug metabolism-related genes indicates that the cultured hepatocytes on the present invention disclosed substrate lead to better function and can be used for high throughput drug discovery and drug safety assessment.

In FIGS. 11-17, 20, 21, 23, and 24, macroarrays 190, microarrays 290, projections 200, and space 390 between microarrays are shown and labeled for purposes of clarity.

Thus, embodiments of SPACED PROJECTION SUBSTRATES AND DEVICES FOR CELL CULTURE are disclosed. One skilled in the art will appreciate that the cell culture apparatuses and methods described herein can be practiced with embodiments other than those disclosed. The disclosed embodiments are presented for purposes of illustration and not limitation.

What is claimed is:

1. An article for culturing cells, comprising:
a substrate having a base surface; and
an array of projections extending from the base surface,
wherein the projections have a height of between about 1 micrometer and about 100 micrometers, and
wherein a gap distance along the base surface from center to center between neighboring projections is between about 10 micrometers and about 80 micrometers.

2. An article according to claim 1, wherein the article comprises a plurality of arrays of projections extending from the base surface.

3. An article according to claim 2, wherein the each array occupies a surface area of the base surface of between about 10,000 square micrometers and about 25,000,000 square micrometers.

4. An article according to claim 2, wherein each of the arrays occupy a generally circular surface area of the base surface having a diameter of between about 100 micrometers and about 500 micrometers.

5. An article according to claim 2, wherein the plurality of arrays occupy between about 10% and about 100% of the surface area of the base surface.

6. An article according to claim 1, wherein the projection are solid.

7. An article according to claim 1, wherein the projection are porous.

8. An article according to claim 1, wherein the base surface of the substrate is the top surface of a bottom of a well of the article.

9. An article according to claim 1, wherein the article consists essentially of a polymeric sheet comprising the array of projects.

10. An article according to claim 1, wherein the projections are formed from polydimethylsiloxane.

11. An article according to claim 1, wherein the article is a 96 well microplate, a 384 well microplate, or 1536 well microplate where each well has a single projection microarray.

12. The article according to claim 11, wherein the gap distance between projections is between 10 μm and 80 μm.

13. A method for culturing functional hepatocyte cells, comprising:
culturing the hepatocyte cells on an article according to claim 1 to restore metabolism functionality of hepatocyte cells.

14. A method according to claim 13, wherein the hepatocyte cells comprise human HepG2C3A cells and wherein the gap distance along the major surface from center to center between neighboring projections is about 15 micrometers to 30 micrometers.

15. A method according to claim 13, wherein the hepatocyte cell comprise immortalized FaN-4 cells, and wherein the gap distance along the major surface from center to center between neighboring projections is about 15 micrometers to 40 micrometers.

16. A method according to claim 13, wherein the hepatocyte cells comprise human primary liver cells, and wherein the gap distance along the major surface from center to center between neighboring projections is about 30 micrometers to 60 micrometers.

17. A method for co-culturing a functional hepatocyte cell with a helper cell, comprising:
co-culturing the hepatocyte cell and the helper cell on an article according to claim 1.

18. A method according to claim 17, wherein the hepatocyte cell is cultured on the article for a period of time prior to addition of the helper cell to the culture.

19. A method according to claim 17, wherein the helper cell is cultured on the article for a period of time prior to addition of the hepatocyte cell to the culture.

20. A method according to claim 17, wherein the hepatocyte cell and the helper cell are added to the culture at the same time.

21. A method according to claim 17, wherein the helper cell is a fibroblast cell, a hepatic stellate cell, or a Kupffer cell.

22. A method for culturing functional hepatocyte cells, comprising:
culturing the hepatocyte cells on an article having a plurality of spaced projections extending from the base surface, wherein the projections are spaced such that at least a portion of the cultured hepatocyte cells contact both the base surface and a surface of a projection.

23. A method for co-culturing a functional hepatocyte cell with helper cells, comprising:
co-culturing the hepatocyte cell and the helper cells on a base surface of an article having a plurality of arrays of spaced apart projections extending from the base surface,
wherein the projections are spaced such that at least a portion of the cultured hepatocyte cells contact both the base surface and a surface of a projection, and
wherein the arrays are spaced apart such that sufficient space is provided for the helper cells to grow on the base surface between the projection arrays.
